(19) United States
(12) Patent Application Publication
(10) Pub. No.: US 2021/0113659 A1
(43) Pub. Date: Apr. 22, 2021

(54) ENGINEERED THERAPEUTIC TO ENHANCE CYTOTOXIC ACTIVITY AND PHAGOCYTOSIS IN TUMOR ASSOCIATED MACROPHAGES

(71) Applicant: University of Massachusetts, Boston, MA (US)

(72) Inventors: Ashish Kulkarni, Arlington, MA (US); Anujan Ramesh, Amherst, MA (US)

(21) Appl. No.: 17/077,041

(22) Filed: Oct. 22, 2020

Related U.S. Application Data
(60) Provisional application No. 62/924,418, filed on Oct. 22, 2019.

(51) Int. Cl. A61K 38/17; C07K 16/28; A61K 31/497; A61K 31/4439; A61K 47/60; A61K 9/51; A61K 45/06; A61K 47/68

(52) U.S. Cl. CPC A61K 38/1774; C07K 16/2851; A61K 31/497; A61K 31/4439; A61K 47/6849; A61K 9/5123; A61K 45/06; A61K 47/6803; A61K 47/60

(57) ABSTRACT

Provided herein are compositions and methods to treat cancer using quantum dynamic capabilities to optimize therapeutic design and development for cancer therapeutics.

### PRIORITY

This application claims the benefit of priority from U.S. Provisional Patent Application Ser. No. 62/924,418, filed on Oct. 22, 2019, which is herein incorporated in its entirety by reference.

### BACKGROUND OF THE INVENTION

Among the plethora of adaptive and innate immune cells recruited to the tumor microenvironment, macrophages are of particular significance due to their relatively high abundance and detrimental role in the onset and maintenance of tumor progression.^{1,2} Tumor associated macrophages (TAMs) are broadly classified into two phenotypes based on their functional states driven by pathogen or cytokine stimulations, the classically activated anti-tumorigenic M1 phenotype and the alternatively activated pro-tumorigenic M2 phenotype.³

### SUMMARY OF THE INVENTION

Provided herein are compositions and methods to treat cancer using quantum dynamic capabilities to optimize therapeutic design and development for cancer therapeutics. Quantum techniques for drug design with molecular simulations confer tumor selectivity and stability to oncology therapeutics that are de novo engineered from a chemical backbone with known binding affinity.

One embodiment provides a composition comprising quantum engineered nanoparticles, wherein the nanoparticles comprise at least one inhibitor of the CSF1-R signaling pathway and at least one inhibitor of the CD47-SIRPα signaling pathway. The inhibitor of CSF1R signaling pathway is engineered de novo from chemical backbone with known binding affinity.

Multivalent protein interactions are quantum engineered, wherein the engineered CD47 protein (see, for example, Gen Bank accession number CEJ95640, the sequence of which is incorporated herein by reference) binds to SIRPα for inhibition of the CD47-SIRPα signaling pathway. The CD47 protein, due to its lower molecular weight as compared to an antibody that binds to SIRP-alpha, can be conjugated to amphiphilic backbone to enable multivalent interactions, higher tumor accumulation and enhanced binding to SIRP-alpha protein. This allows for smaller antigen sink, lower dosing in patients, lack of RBC binding, and low to no anemia.

In one embodiment, the engineered nanoparticles comprise phosphatidyl choline (PC), such as L-α-phosphatidylcholine. In another embodiment, the engineered nanoparticles comprise cholesterol. In one embodiment, the engineered nanoparticle is pegylated, such as by 1,2-distearoyl-sn-glycero-3-phosphoethanolamine-N-[amino(polyethylene glycol)-2000]. In one embodiment, the at least one inhibitor of the CSF1-R signally pathway is CSF1R kinase inhibitor, such as 4-((2-(((1R,2R)-2-hydroxycyclohexyl)amino)benzo[d]thiazol-6-yl)oxy)-N-methylpicolinamide (BLZ-945). In one embodiment, the at least one inhibitor of the CD47-SIRPα signaling pathway is an inhibitor of SHP2, such as 6-(4-Amino-4-methylpiperidin-1 -yl)-3-(2,3-dichlorophenyl)pyrazin-2-amine dihydrochloride (SHP099). In embodiment, the engineered nanoparticle is conjugated to a targeting agent, such as an anti-CD206 antibody. In one embodiment, the composition further comprises a carrier.

One embodiment provides a method to treat cancer comprising administering to a subject in need thereof the composition described herein. In one embodiment, the method further comprises administering an additional anti-cancer agent, such as chemotherapy, radiation or immunotherapy.

One embodiment provides a method to repolarize M2 macrophages to activated M1 macrophages comprising contacting the M2 macrophage with a composition as described herein. Another embodiment provides a method to increase phagocytic function of a macrophage comprising contacting the macrophage with a composition described herein.

### BRIEF DESCRIPTION OF THE DRAWINGS

FIGS. 1A-B. Schematics highlighting the mechanism of dual inhibition by DNTs: (A) Schematics show that simultaneous activation of the CSF1-R pathway and the CD47-SIRPα pathway by cancer cells resulting in M2 polarized pro-tumorigenic macrophages. (B) Schematics show deterministic co-delivery of DNTs to the M2 polarized macrophage leads to concurrent inhibition of CSF1-R and SHP2 which results in repolarization of macrophages to an anti-tumorigenic M1 phenotype while simultaneously increases the phagocytic index.

FIGS. 2A-J. DNT synthesis and characterization: (A) Schematics showing the synthesis of DNTs using thin-film hydration method. CSF-1R inhibiting amphiphile and SHP099 were loaded into stable supramolecular therapeutics facilitated by self-assembly of co-lipids (DSPE PEG and PC). (B) High-resolution cryo-TEM image of DNTs showing an average size of ˜90 nm and having a spherical morphology (C) Graph shows stability of DNTs as a function of change in size distribution and zeta potential over time, measured by Dynamic Light Scattering during storage condition at 4° C. (D-E) Graphs show stability of DNTs incubated in human serum as a function of change in size distribution and zeta potential over time, measured by Dynamic Light Scattering (F-G) Drug release profiles of iCSF1-R and iSHP2 from DNTs when incubated in macrophage cell lysate (pH ˜5.5) and PBS (pH 7.4) (H-I) Graphs show amount of drug internalized into M2 macrophages after 18 h incubation as quantified by UV-Vis Spectroscopy compared to free drug. Data shown are mean±s.e.m. (n=3). Statistical analysis was performed with student t-test. *p<0.05, ****p<0.0001. (J) Representative confocal images for internalization of FITC-tagged BSNPs into M2 polarized macrophages after 4 h incubation compared to control. The nuclei were DAPI stained and the cell surface was stained with Alexa Fluor 594 F480.

FIGS. 3A-F. In vitro efficacy studies of DNTs: (A) Schematic representation of the repolarization assay. RAW264.7 macrophages were polarized to M2 phenotype by treatment with IL-4 for 24 h, following which treatments were added in fresh medium for 48 h and sample was collected for FACS and western blotting (B-C) Graphs demonstrating expression of M2 (CD11b⁺, CD206⁺) and M1/M2 ratio (CD11b⁺, CD80)/(CD11b⁺CD206⁺) respectively on macrophages as quantified from flow cytometry at 48 h following different treatments of 500 nM concentration. Data shown are mean±s.e.m. (n=3). Statistical analysis was performed with one-way ANOVA followed by Newman-Keuls posttest. ns—not significant, **p<0.01, ***p<0.001, ****p<0.0001. (D) Western Blots showing the expression of SHP-2, phosphor CSF1R and Total CSF1R in macrophages subjected to different treatments at different time points. (E) Schematic representation of phagocytosis assay. RAW264.7 macrophages were stained with Cell Trace Far Red and incubated with different treatments for 48 h. Following this, the cancer cells were stained with Cell Trace CFSE and co-cultured with treated macrophages for 4 h and FACS was performed. (F) Graphs show percentage of macrophages that are phagocytic in a co-culture of RAW264.7 with 4T1 breast cancer. Data shown are mean±s.e.m. (n=3). Statistical analysis was performed with one-way ANOVA followed by Newman-Keuls post-test. ns—not significant, **p<0.01.

FIGS. 4A-G. In vivo efficacy studies of DNTs in immunocompetent 4T1 breast cancer model: (A) Schematic showing the dosage schedule of DNT treatments. (B) Graph shows the tumor growth profiles of mice treated with DNTs and other treatments. Data shown are mean±s.e.m. (n=5). Statistical significance was determined using one-way ANOVA with Newman-Keuls post-test **p<0.01. (C) The toxicity of each treatment group was assessed by measuring the changes in overall body weight. (n=5, for each treatment group) (D-E) Graphs quantifying the expression of different effector T-cell markers (CD3⁺CD8⁺, CD3⁺CD4⁺) from a single cell suspension of excised tumors. Data shown are mean±s.e.m. (n=3). Statistical significance was determined using one-way ANOVA with Newman-Keuls post-test. **p<0.01, ****p<0.0001. (F-G) Graphs show quantification of M1 (CD45⁺CD11b⁺CD80) and M2 (CD45⁺CD11b⁺CD206) markers in the tumor samples from different treatment groups. Data shown are mean±s.e.m. (n=3). Statistical significance was determined using one-way ANOVA with Newman-Keuls post-test. ns—not significant, *p<0.05, ****p<0.0001.

FIGS. 5A-F. Mechanistic analysis of efficacy of DNTs: (A) Representative confocal images of tumor sections obtained from different treatment groups were stained with CD45 (red) and CD8a (green) to identify infiltrating CD8⁺ cytotoxic T cells. The sections were counterstained with DAPI (blue). (B) Representative confocal images of tumor sections obtained from different treatment groups. The sections were stained using TUNEL (red) to identify apoptosis and counterstained with DAPI (blue). (C) Graph shows quantification of total apoptotic cells in tumor sections obtained from different treatment groups. Total TUNEL positive cells were counted (Red) per 100 DAPI cells (Blue). Data shown are mean±s.e.m. (n=3). Statistical significance was determined using one-way ANOVA with Newman-Keuls post-test. **p<0.01. (D) Representative western blots obtained from tumor lysates of mice subjected to different treatments showing expression of SHP2. Phopho-SHP2, Total CSF1-R, phospho-CSF1R, Total ERK1/2, Phopho-ERK1/2, IFNγ. (E-F) Graphs showing the quantification of SHP2 and phospho CSF1-R expression in tumor lysates obtained from mice subjected to different treatments. Data shown are mean±s.e.m. (n=3). Statistical significance was determined using one-way ANOVA with Newman-Keuls post-test. *p<0.05, ***p<0.001, ****p<0.0001.

FIGS. 6A-E. In vivo efficacy studies of DNTs in immunocompetent B16F10 melanoma model: (A) Schematic showing the dosage schedule of DNT treatments. (B) Graph shows the tumor growth profiles of mice treated with DNTs and other treatments. Data shown are mean±s.e.m. (n=5). Statistical significance was determined using one-way ANOVA with Newman-Keuls post-test *p<0.05, **p<0.01. (C) The toxicity of each treatment group was assessed by measuring the changes in overall body weight. (n=5, for each treatment group). (D) Graph represents the activity of intertumoral Granzyme B in tumors obtained from mice post treatment. The harvested tumors were lysed, and the amount of Granzyme B was estimated using a Granzyme B quantification kit. (E) Representative confocal images showing cross sections of tumors excised from mice. The cross sections were stained with a cancer cell marker (CD47) and a macrophage marker (CD11b). Phagocytic macrophages are represented by cells which are dual stained for both CD47 and CD11b. Statistical significance was determined using one-way ANOVA. **p<0.001.

FIGS. 7A-B. (A) Representative size distribution graph of free drug loaded nanoparticles after 10 mins of synthesis. Insert shows free drug loaded nanoparticles are highly unstable and aggregate within 10 mins of synthesis. (B) Representative size distribution graphs of DNTs depicting the hydrodynamic diameter after 72 h of synthesis. Insert shows that the DNTs remain stable for extended periods of time.

FIGS. 8A-B. (A) Graph shows optimized loading of BLZ945 and SHP099 in DNTs. DNTs were synthesized using the lipid film hydration technique incorporating the dual inhibitors. DNTs were then passed through a sephadex column to remove free inhibitors. The concentration of inhibitors was quantified by UV-Vis Spectroscopy. Data shown are mean±s.e.m. (n=3). (B) Graph shows relationship between increasing concentrations of cholesterol and SHP099 loading in SHP-SNP. Data shown are mean±s.e.m. (n=2). Statistical significance was determined using one-way ANOVA with Newman-Keuls post-test. ns—not significant.

FIG. 9. Schematics showing the synthesis of the CSF1-R inhibiting amphiphile.

FIGS. 10A-B. (A) Representative flow cytometry figures for quantification of apoptosis and necrosis of RAW 264.7 macrophages following treatment with increasing concentrations of either DNTs or equimolar free drug combinations. (B) Graph shows the toxicity of DNTs in comparison to equimolar free drug combination, measured by Annexin V/PI Assay. Data shown are mean±s.e.m. (n=3).

FIG. 11. Graphs demonstrating expression of the M2 marker (CD11b⁺C163⁺) on macrophages as quantified from flow cytometry at 48 h following different treatments of 500 nM concentration. Data shown are mean±s.e.m. (n=3). Statistical analysis was performed with one-way ANOVA followed by Newman-Keuls post-test. ns—not significant, ****p<0.0001.

FIGS. 12A-B. (A) Representative confocal images showing cells subjected to various treatments stained for Nitric oxide (NO) signal. (B) Graph showing the quantification of number of cells secreting Nitric Oxide per 100 DAPI+ve cells. Statistical analysis was performed with one-way ANOVA with Newman-Keuls post-test. Data show mean±s.e.m.(n=3). ****p<0.001.

FIG. 13. Representative flow cytometry plots showing expression of M1 (CD11b⁺, CD80⁺) and M2 markers (CD11b⁺, CD206⁺) in macrophages treated with different treatment groups at 500 nM concentration (equivalent of each free drugs).

FIGS. 14A-C. (A) Schematic representation of the repolarization assay. RAW264.7 macrophages were polarized to M2 phenotype by treatment with IL-4 for 24 h, following which treatments were added in fresh medium for 12 h and sample was collected for FACS and western blotting. (B-C) Graphs demonstrating expression of M2 (CD11b⁺, CD206⁺) and M1 (CD11b⁺, CD80⁺) markers respectively on macrophages as quantified from flow cytometry at 12 h following different treatments of 500 nM concentration. Statistical analysis was performed with one-way ANOVA followed by Newman-Keuls post-test. ns—not significant, ***p<0.001.

FIGS. 15A-B. Graphs showing the repolarization efficiency as represented by the M1/M2 ratio (CD11b⁺, CD80)/(CD11b⁺CD206⁺). Expression levels were quantified using flow cytometry at 48 h following different treatments of 500 nM concentration. Data shown are mean±s.e.m. (n=3). Statistical analysis was performed with one-way ANOVA followed by Newman-Keuls post-test. ns—not significant, *p<0.05, **p<0.01.

FIGS. 16A-B. (A) Schematics representing the isolation of peripheral blood monocytes from whole blood and its differentiation into M2 macrophages. Briefly PBMCs were separated from whole blood using a Ficoll gradient. Isolated PBMCs were made to attach onto petri-plates in the presence of human MCSF. After 7 days the attached macrophages were then treated with human IL-4 to obtain M2 differentiated macrophages. (B) Graphs demonstrating the M1/M2 ratio (CD11b⁺, CD80)/(CD11b⁺CD206⁺) on PBMC derived macrophages. Expression levels were quantified using flow cytometry at 48 h following different treatments of 500 nM concentration. Data shown are mean±s.e.m. (n=3). Statistical analysis was performed with one-way ANOVA followed by Newman-Keuls post-test., **p<0.01, ***p<0.001.

FIGS. 17A-B. (A) Graphs demonstrating expression of the M1/M2 ratio (CD11b⁺, CD80)/(CD11b⁺CD206⁺) respectively on Bone marrow derived macrophages as quantified from flow cytometry at 48 h following different treatments of 500 nM concentration. Data shown are mean±s.e.m. (n=3). Statistical analysis was performed with one-way ANOVA followed by Newman-Keuls post-test. **p<0.01 (B) Graphs show percentage of macrophages that are phagocytic in a co-culture of RAW264.7 with 4T1 breast cancer and. Data shown are mean±s.e.m. (n=3). Statistical analysis was performed with one-way ANOVA followed by Newman-Keuls post-test. *p<0.05, ***p<0.001.

FIGS. 18A-B. (A) Schematic representation of phagocytosis assay. RAW264.7 macrophages were stained with Cell Trace Far Red and incubated with different treatments for 48 h. Following this, the cancer cells were stained with Cell Trace CFSE and co-cultured with treated macrophages for 4 h and FACS was performed. (B) Graphs show percentage of macrophages that are phagocytic in a co-culture of RAW264.7 with B16F10 melanoma cancer cells. Data shown are mean±s.e.m. (n=3). Statistical analysis was performed with one-way ANOVA followed by Newman-Keuls post-test. ns—not significant, ***p<0.001.

FIGS. 19A-D. (A) Representative NiR images showing time dependent accumulation of NiR-tagged DNTs on tumor bearing mice. The tumor is represented by dotted lines. (B) Graph shows quantification of time dependent accumulation of NiR tagged DNT in tumor bearing mice (C) Representative NiR images of organs excised from mice after 24 h of NiR-tagged DNT injection. (D) Graph shows quantification of DNT accumulation in excised organs after 24 h of DNT injection. Data show mean±s.e.m.(n=3). Statistical analysis was performed with one-way ANOVA with Newman-Keuls post-test. **p<0.01.

FIGS. 20A-B. (A) Representative confocal images showing cross sections of tumors excised from mice, stained for pan macrophage marker CD11b.Tumor bearing mice were intravenously administered with either FITC tagged DNTs or control DNTs and sacrificed after 24 h. (B) Graph showing the quantification of number of macrophages that have internalized DNTs per total macrophages Statistical analysis was performed with one-way ANOVA with Newman-Keuls post-test. Data show mean±s.e.m. (n=3). ***p<0.001.

FIGS. 21A-D. (A) Graph shows the effect of increasing concentrations of DNTs and BLZ+SHP FD on the viability of B16F10 melanoma cells. (B) Table shows the calculated IC50 values of DNTs and BLZ+SHP FD after 72 h of treatment on B16F10 melanoma cells. (C) Graph shows the effect of increasing concentrations of DNTs and BLZ+SHP FD on the viability of 4T1 breast cancer cells line. (D) Table shows the calculated IC50 values of DNTs and BLZ+SHP FD after 72 h of treatment on 4T1 breast cancer cells.

FIGS. 22A-B. (A) Representative confocal images showing cross sections of liver excised from mice, stained for apoptosis signal (TUNEL). (B) Graph showing the quantification of number of apoptotic cells per 100 DAPI cells. Quantification was done by counting the number of TUNEL+ve cells stained red and total DAPI cells stained blue. Statistical analysis was performed with one-way ANOVA with Newman-Keuls post-test. Data show mean±s.e.m. (n=3). ns—not significant.

FIGS. 23A-C. (A) Schematic representation shows conjugation of anti-CD206 nanobodies to the surface of DNT-206 facilitated by maleimide-thiol reaction. (B) Representative size distribution graph of DNT-206 measured by Dynamic Light Scattering. (C) Graph shows stability of DNT-206 as a function of change in size distribution and zeta potential over time, measured by Dynamic Light Scattering during storage condition at 4° C.

FIGS. 24A-B. (A) Quantification of flow cytometry plots showing no significant difference in the % M1 and M2 macrophage composition between the two groups. Data shown are mean±s.e.m. (n=3). Statistical significance was determined using one-way ANOVA with Newman-Keuls post-test. (B) Graph showing % macrophages that are phagocytotic between DNTs and DNT-206. No significant difference is observed between them. Data shown are mean s.e.m. (n=3). Statistical significance was determined using one-way ANOVA with Newman-Keuls post-test. ns—not significant.

FIGS. 25A-D. (A-C) Quantification of expression of effector T cell and NK cell markers (CD45⁺CDS⁺, CD45⁺CD4⁺, CD45⁺NK1.1⁺) in a single cell suspension of harvested tumor post treatments. Data shown are mean±s.e.m. (n=3), Statistical significance was determined using one-way ANOVA with Newman-Keuls post-test. *p<0.05, **p<0.01. (d) Quantification of the ratio of M1 macrophages (CD45⁺CD11b⁺CD86⁺) as compared to M2 macrophages (CD45⁺CD11b⁺CD206⁺) in a single cell suspension of harvested tumor post treatments. Data shown are mean±s.e.m. (n=3), Statistical significance was determined using one-way ANOVA with Newman-Keuls post-test. ns—not significant, *p<0.05.

FIGS. 26A-B. (A) Representative confocal images showing cross sections of tumors excised from mice, stained for apoptosis signal (TUNEL). (B) Graph showing the quantification of number of apoptotic cells per 100 DAPI cells. Quantification was done by counting the number of TUNEL+ve cells stained red and total DAPI cells stained blue. Statistical analysis was performed with one-way ANOVA with Newman-Keuls post-test. Data show mean±s.e.m.(n=3). ns—not significant, ****p<0.0001.

FIG. 27. Flow cytometry gating strategy for ex vivo analysis.

FIGS. 28A-B. (A) Representative flow cytometry plots show that the engineered CD47 binds to SIRP-alpha on the macrophage surface and reduces the amount of SIRP-alpha available for binding to FITC-dye-tagged anti-SIRP-alpha antibody. (B) Graphs show percentage of macrophages that are phagocytic after different treatments in a co-culture of RAW264.7 with B16F10 melanoma cells. RAW264.7 macrophages were stained with Cell Trace Far Red and incubated with different treatments for 48 h. Following this, the cancer cells were stained with Cell Trace CFSE and co-cultured with treated macrophages for 4 h and FACS was performed. Treatment with multivalent- CD47 results in significant enhancement in phagocytic ability of macrophages as compared to other treatment. Data shown are mean±s.e.m. (n=3). Statistical analysis was performed with one-way ANOVA followed by Newman-Keuls post-test. ns—not significant, **p<0.01.

### DETAILED DESCRIPTION OF THE INVENTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, several embodiments with regards to methods and materials are described herein. As used herein, each of the following terms has the meaning associated with it in this section.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The articles “a” and “an” are used herein to refer to one or to more than one (i.e. to at least one) of the grammatical object of the article. By way of example, “an element” means one element or more than one element.

The phrase “and/or,” as used herein, should be understood to mean “either or both” of the elements so conjoined, e.g., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, e.g., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

The terms “individual,” “subject,” and “patient,” are used interchangeably herein and refer to any subject for whom diagnosis, treatment, or therapy is desired, including a mammal. Mammals include, but are not limited to, humans, farm animals, sport animals and pets. A “subject” is a vertebrate, such as a mammal, including a human. Mammals include, but are not limited to, humans, farm animals, sport animals and companion animals. Included in the term “animal” is dog, cat, fish, gerbil, guinea pig, hamster, horse, rabbit, swine, mouse, monkey (e.g., ape, gorilla, chimpanzee, orangutan) rat, sheep, goat, cow and bird.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease.

The terms “inhibit”, “inhibiting”, and “inhibition” refer to the slowing, halting, or reversing the growth or progression of a disease, infection, condition, group of cells, protein or its expression. The inhibition can be greater than about 20%, 40%, 60%, 80%, 90%, 95%, or 99%, for example, compared to the growth or progression that occurs in the absence of the treatment or contacting.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 20% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” is an amount sufficient to effect beneficial or desired result, such as preclinical or clinical results. An effective amount can be administered in one or more administrations.

The terms “cell,” “cell line,” and “cell culture” as used herein may be used interchangeably. All of these terms also include their progeny, which are any and all subsequent generations. It is understood that all progeny may not be identical due to deliberate or inadvertent mutations.

As used herein, an “instructional material” includes a publication, a recording, a diagram, or any other medium of expression which can be used to communicate the usefulness of the ligand-drug conjugate, such as a peptide ligand, of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

As used herein, the term “pharmaceutically-acceptable carrier” means a chemical composition with which an appropriate compound or derivative can be combined and which, following the combination, can be used to administer the appropriate compound to a subject. “Pharmaceutically acceptable” means physiologically tolerable, for either human or veterinary application. As used herein, “pharmaceutical compositions” include formulations for human and veterinary use.

By the term “specifically binds to”, as used herein, is meant when a compound or ligand functions in a binding reaction or assay conditions which is determinative of the presence of the compound in a sample of heterogeneous compounds, or it means that one molecule, such as a binding moiety, e.g., an oligonucleotide or antibody, binds preferentially to another molecule, such as a target molecule, e.g., a nucleic acid or a protein, in the presence of other molecules in a sample..

The terms “specific binding” or “specifically binding” when used in reference to the interaction of a peptide (ligand) and a receptor (molecule) also refers to an interaction that is dependent upon the presence of a particular structure (e.g., an amino sequence of a ligand or a ligand binding domain within a protein); in other words the ligand comprises a structure allowing recognition and binding to a specific protein structure within a binding partner rather than to molecules in general. For example, if a ligand is specific for binding pocket “A,” in a reaction containing labeled peptide ligand “A” (such as an isolated phage displayed peptide or isolated synthetic peptide) and unlabeled “A” in the presence of a protein comprising a binding pocket A the unlabeled peptide ligand will reduce the amount of labeled peptide ligand bound to the binding partner, in other words a competitive binding assay.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

Methods involving conventional molecular biology techniques are described herein. Such techniques are generally known in the art and are described in detail in methodology treatises, such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22: 1859-1862, 1981, and Matteucci et al., J. Am. Chem. Soc. 103:3185, 1981.

As used herein, the terms “including”, “includes”, “having”, “has”, “with”, or variants thereof, are intended to be inclusive similar to the term “comprising.”

The terms “comprises,” “comprising,” and the like can have the meaning ascribed to them in U.S. Patent Law and can mean “includes”, “including” and the like. As used herein, “including” or “includes” or the like means including, without limitation.

### Overview of Composition/Methods

Macrophages are one of the most abundant cells of the tumor stroma and are found to be involved in various stages of disease progression. Due to its high numbers and inherent plasticity, immune modulation of macrophages has emerged as an attractive approach for anti-cancer therapy. However, there are two main challenges in successfully utilizing macrophages for immunotherapy. First, macrophage colony stimulating factor (MCSF) secreted by cancer cells binds to Colony stimulating factor 1 receptor (CSF1-R) on macrophages and in turn activates the downstream signaling pathway responsible for polarization of TAMs to alternatively activated M2 phenotype. These immunosuppressive M2 macrophages release pro-tumorigenic factors that dampen the anti-tumor immune response. Second, myeloid cells including monocytes express a signal regulatory protein called SIRPα that binds to CD47, a transmembrane protein overexpressed on cancer cells. This ligation activates the Src homology region 2 (SH2) domain -phosphatases SHP-1 and SHP-2 in macrophages resulting in activation of “eat-me-not” signaling pathway and inhibition of phagocytosis. Hence, therapeutic strategies aimed at switching macrophage phenotype from a pro-tumorigenic M2 to an anti-tumorigenic M1 through CSF1R signaling inhibition while simultaneously blocking CD47-SIRPα axis to enhance phagocytosis offer a better approach to macrophage modulation. However, sub-par response rates coupled with high systemic toxicities observed in individual anti-CSF1R and anti-CD47 treatment strategies calls for new approaches for efficient inhibition of both pathways at the same time. Here, it is reported that self-assembled dual inhibitor loaded nanoparticles (DNTs) target M2 macrophages and simultaneously inhibit CSF1R and SHP2 pathways. This results in efficient repolarization of M2 macrophages to an active M1 phenotype, and superior phagocytic capabilities as compared to individual drug treatments. Furthermore, sub-optimal dose administration of DNTs in highly aggressive breast cancer mouse model showed enhanced anti-tumor efficacy without any toxicity. The mechanistic analysis of efficacy revealed surprising and unexpected immune activation in the tumor in terms of enhanced repolarization of TAMs to M1 phenotype, increased phagocytosis efficacy and significantly higher infiltration of effector T cells in DNT treated tumors as compared to free drug treatments. Targeted delivery of both inhibitors using anti-CD206 antibody conjugated DNTs resulted in enhanced efficacy in aggressive melanoma tumor model. It is also reported that the multivalent CD47 protein can efficiently inhibit CD47-SIRPalpha signaling and result in enhanced phagocytosis potential of macrophages. The lower molecular weight of CD47 protein imparts additional advantages as compared to full antibodies such as higher tumor penetration and accumulation.

### I. Delivery System/Nanoparticles and Nanoparticle Materials

The delivery vehicle can be nanoparticles, microparticles, liquid crystals, liposomes, protein complexes, self-assembled supramolecular systems or molecules. The delivery vehicles may consist of any material or combination of materials such as proteins such as albumin and/or ferritin, phospholipids such as dioleylphosphateidylethanolamine, phosphatidyl choline (PC), including L-α-phosphatidylcholine, and/or dioleylphosphatidylcholine, 1,2-dioleoyl-sn-glycero-3-phosphocholine, dipalmitoylphosphatidylcholine, 1,2-Dimyristoyl-sn-glycero-3-phosphocholine, 1,2-dioleoyl-sn-glycero-3-phospho-(1′-rac-glycerol), polymers such as poly(lactic-co-glycolic) acid, poly(lactic acid), polyethylene glycol, polyethylenimine, polymethylmethacrylate, polyhydroxyalkanoate, triblock copolymers of poly(ethylene oxide) and/or poly(propylene oxide), and/or inorganic materials such as gold and/or silica. The nanoparticles can also be pegylated, such as by 1,2-distearoyl-sn-glycero-3-phosphoethanolamine-N-[amino(polyethylene glycol)-2000].

### II. Cell Targeting Agents for DNTs

Targeting agents can include, but are not limited to, antibodies, nanobodies, aptamers, RNA, DNA, proteins, polymers, oligomers, monomers, and surfactants, that target, for example, cells (cancer antigens) or immune cells surface receptors. Targeting agents can be any particle, or simple or complex molecule, used to target any cell, such as an antibody. In one embodiment, the targeting agent is anti-CD206 antibody. In one embodiment, the targeting agents are conjugated to the DNTs.

### III. Inhibitors

The inhibitors present in the nanoparticles are inhibitors of the CSF1-R, CD47-SIRPα signaling pathways and scavenger receptors. At least one inhibitor for each pathway is present in the nanoparticles. In one embodiment, the inhibitor of the CSF1-R signaling pathway is an inhibitor of CSF-1R kinase, such as 4-((2-(((1R,2R)-2-hydroxycyclohexyl)amino)benzo[d]thiazol-6-yl)oxy)-N-methylpicolinamide. In another embodiment, the inhibitor of the CD47-SIRPα signaling pathway is an inhibitor of SHP-2, including 6-(4-Amino-4-methylpiperidin-1-yl)-3-(2,3-dichlorophenyl)pyrazin-2-amine dihydrochloride (SHP099). The amphiphilic inhibitors of CSF1R include conjugates of BLZ945, PLX3397, PLX5622, cFMS Receptor Inhibitor II, AC710, ARRY-382, Edicotinib, TN0155 with cholesterol, Sitosterol, 1-Lysophosphatidylcholine with either of hydrophobic or hydrophilic linkers.

### IV. Cancer Treatment

Types of cancer that can be treated using the methods of the invention include, but are not limited to, solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

### V. Administration

The invention includes administration of nanoparticles described herein and methods for preparing pharmaceutical compositions and administering such as well. Such methods comprise formulating a pharmaceutically acceptable carrier with the nanoparticles described herein.

A pharmaceutical composition of the invention is formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic (such as lyophilized bacteria and bacteria thawed from frozen solution).

For intravenous administration, suitable carriers include physiological saline, bacteriostatic water, CremophorEL™ (BASF; Parsippany, N.J.) or phosphate buffered saline (PBS). It must be stable under the conditions of manufacture and storage and must be preserved against the contaminating action of microorganisms. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Injectable solutions can be prepared by incorporating the active compound (or DNTs) in the required amount in an appropriate solvent with one or a combination of ingredients discussed above. Generally, dispersions are prepared by incorporating the active compound into a vehicle which contains a basic dispersion medium and various other ingredients discussed above. In the case of powders for the preparation of injectable solutions, methods of preparation are vacuum drying and freeze-drying which yields a powder of the active ingredient plus any additional desired ingredient from a previously.

Oral compositions generally include an inert diluent or an edible carrier. For example, they can be enclosed in gelatin capsules. For the purpose of oral therapeutic administration, the active compound/DNT can be incorporated with excipients and used in the form of tablets, troches, or capsules.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

For administration by inhalation, the nanoparticles are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the bacteria are formulated into ointments, salves, gels, or creams as generally known in the art.

It can be advantageous to formulate compositions in dosage unit form for ease of administration and uniformity of dosage. Dosage unit form as used herein refers to physically discrete units suited as unitary dosages for the subject to be treated; each unit containing a predetermined quantity of active compound/DNT calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

### EXAMPLE

The following example is provided in order to demonstrate and further illustrate certain embodiments and aspects of the present invention and are not to be construed as limiting the scope thereof.

### Example I

### Introduction

Among the plethora of adaptive and innate immune cells recruited to the tumor microenvironment, macrophages are of particular significance due to their relatively high abundance and detrimental role in the onset and maintenance of tumor progression.^{1,2} Tumor associated macrophages (TAMs) are broadly classified into two phenotypes based on their functional states driven by pathogen or cytokine stimulations, the classically activated anti-tumorigenic M1 phenotype and the alternatively activated pro-tumorigenic M2 phenotype.³ The tumor microenvironment is endowed with an assortment of immunosuppressive cytokines involved in polarizing TAMs to an “M2-like lineage”, which helps in advancing metastasis, angiogenesis and also suppresses the activity of other effector cells like CD8⁺ and CD4⁺T cells.^{4,5} Recent clinical evidence further reiterates the fact that presence of tumor associated macrophages have been positively linked with poor prognosis in cancers such as breast cancer and melanoma.⁶⁻¹¹ However, one can effectively exploit the inherent plasticity of macrophages by “re-educating” TAMs from an M2 phenotype to anti-tumorigenic M1 phenotype. To realize this goal, therapeutic strategies aimed at targeting signaling pathways involved in macrophage activation are needed. For instance, the MCSF-CSF1-R pathway is activated upon binding of MCSF to the CSF1-receptors expressed on myeloid cells. Presence of MCSF in the tumor microenvironment promotes recruitment and differentiation of TAMs into an immunosuppressive M2 like phenotype.¹² However, recent clinical trials have shown that single agent therapies (monoclonal antibodies or small molecule inhibitors) targeting CSF1R signaling axis have limited efficacy.¹³ Clinical trials involving Pexidartinib, ARRY-382, MCS11 showed underwhelming results in terms of anti-cancer efficacy owing to poor response rates.¹⁴⁻¹⁸ Furthermore, recent studies have emphasized the importance of sustained inhibition of the CSF1-R axis in order to continually maintain macrophages in an activated M1 state.¹⁸⁻²¹ Another major challenge faced by CSF1-R inhibiting strategies is the fact that CD47, a membrane protein expressed on the surface of most solid tumors, is a player in regulating macrophage phagocytosis.₂₂ Engagement of CD47 with SIRPα, a transmembrane protein expressed on the surface of myeloid cells initiates a downregulatory “eat me not” signal that inhibits the phagocytic functionality of the host macrophages, thus making CSF1-R inhibition ineffective.²³ Phase I clinical trials involving single agent administration of monoclonal antibodies blocking the CD47-SIRPα include humanized CD47-blocking agents like Hu5F9-G4, CC-90002 and SIRPα protein variants like TTI-621 and ALX148.²⁴ Clinical trial evidence increasingly points to limited anti-cancer efficacies associated with these therapies owing to limitations such as sub-par binding affinities of SIRPα antibodies and systemic toxicities involving anti-CD47 based therapies.²⁵⁻²⁸

Molecular mechanisms involved in the CD47-SIRPα reveal that upon engagement with CD47, SIRPα functions as a docking protein and activates the Src homology region 2 (SH2) domain-phosphatases SHP-1 and SHP-2. Tyrosine phosphorylation of cytoplasmic regions of SIRPα as a result of activation of SHP-1 and SHP-2 sets off downstream signaling pathways responsible for SIRPα function.²⁹ Here, a rationale strategy of simultaneously activating and re-educating macrophages by CSF1R kinase inhibition and increasing their phagocytosis potential by SHP2 phosphatase inhibition (resulting in suppression of downstream signaling of CD47-SIRPα axis) was explored/presented. A recent study reported that SHP2 inhibitors (SHP099) inhibited the growth of tumor cells through an RTK-SHP2 dependent manner.³⁰ Previous studies have extensively studied the tumor progressive role played by SHP2 in cancer cells, but its role in macrophage functionality has been relatively unexplored.

### Material and Methods

Materials

All the reagents were of analytical grade and used as supplied without further purification unless specified. The reactions were maintained at inert conditions unless otherwise specified. Dichloromethane (DCM), Methanol and N, N-dimethylformamide (DMF) were purchased from Fisher Scientific. L-α-phosphatidylcholine (PC), and Sephadex G-25 were purchased from Sigma-Aldrich. Cholesterol, 1,2-Distearoyl-sn-Glycero-3-Phosphoethanolamine-N-[Carboxy (Polyethylene Glycol)2000] (DSPE-PEG-Carboxylic Acid), mini hand-held extruder kit including the 0.4 μ, 0.2 μm, m Nucleopore Track-Etch Membrane, 10 mm Filter supports and 250 ml Syringes were bought from Avanti Polar Lipids. 1-Ethyl-3-(3-dimethylaminopropyl) carbodiimide (EDC) and N-hydroxysulfosuccinamide (Sulfo-NHS) was purchased from Thermo Scientific. SHP099 and BLZ945 were purchased from Selleckchem.CD11b, CD45, CD8a, CD4, NK1.1, CD86, CD80, CD206 antibodies were purchased from Biolegend Inc. pCSF1R, TCSF1R, pSHP2, SHP2, ERK1/2, pERK1/2 antibodies were purchased from Cell Signaling Technology. Alexa Fluor 594 Goat anti-rabbit IgG and Alexa Flour 488 Goat anti-Rabbit IgG antibody was purchased from Thermo Fisher. 6 wells and 12 wells, 5 mL, and 10 mL plates were purchased from Corning. DMEM, FBS, and antibiotic-antimycotic were purchased from Gibco, Life Technologies. Fluorescence spectra were obtained using a BioTek plate reader. Flow cytometry was performed using ACEA Novoflow Flow Cytometer and data was analyzed using NovoExpress software. Mean particle size and zeta potential were measured by Dynamic Light Scattering method using Malvern Zetasizer Nano ZSP. Cryo-Transmission Electron microscopy was performed using a FEI Tecnai Cryo-Bio 200 KV FEG TEM and confocal microscopic images were obtained with Nikon Al SP Spectra and analyzed using NIS elements software.

Methods

Synthesis of CSF-1R inhibiting amphiphile: BLZ-945 (BLZ) was dissolved in 2 ml anhydrous Dichloromethane (Fisher Scientific) to which 1.1 molar equivalents of cholesterol hemisuccinate, DMAP and EDC was added. The reaction was stirred for 24 hours at room temperature. The reaction was monitored through thin layer chromatography and once the reaction was completed, the crude product was purified using column chromatography, eluting it with a 3% Methanol: DCM gradient to give the CSF-1R inhibiting amphiphile as a yellow solid.

Synthesis and characterization of single-inhibitor loaded nanoparticles and dual-inhibitor loaded nanoparticles: The supramolecular nanoparticles were synthesized using the lipid-film hydration method. 5% of CSF-1R inhibiting amphiphile, 5% of SHP2 inhibitor, 30 mol % of 1,2-distearoyl-sn-glycero-3-phosphoethanolamine-N-[amino (polyethylene glycol)-2000] and 60 mol % L-α-phosphatidylcholine was dissolved in 1 ml of DCM. For single-inhibitor loaded nanoparticles, 10 mol % of either CSF-1R inhibiting amphiphile or SHP2 inhibitor was used. The solvent was evaporated into a thin and uniform film using a rotary evaporator. This thin film was hydrated with PBS for 1 h at 60° C. to obtain self -assembled supramolecular nanoparticles. The nanoparticles were extruded through a 0.4 μμm and 0.2 μμm polycarbonate membrane using a mini extruder and were eluted through a sephadex column. The hydrodynamic diameter was measured using Dynamic Light Scattering (DLS) and the drug loading was determined by UV spectroscopy.

Stability Studies of single-inhibitor loaded, and dual-inhibitor loaded nanoparticles: The amount of inhibitor(s) loaded into the single and dual inhibitor nanoparticles was determined by UV-Vis Spectroscopy. The efficiency of drug loading was determined as the percentage of drug recovered from the nanoparticle fractions compared to the initial loading amount. The physical stability of the nanoparticles was determined by measuring changes in the mean particle size at storage conditions of 4° C. for different time points.

Stability of Nanoparticle in Human Serum: DNTs were first synthesized. To this, 10% human serum was added, and the mixture was stirred gently at 4° C. The mean particle size of the nanoparticles was measured at different time points after serum addition by Dynamic Light Scattering method using Zetasizer Nano Z S90 (Malvern, UK) at regular intervals.

Release kinetics studies: DNTs were suspended in PBS (pH 7.4) or RAW 264.7 macrophage cell lysate (pH ˜5-6) and were sealed in dialysis tubes (MWCO=3500 Dalton, Spectrum Labs). The dialysis tubes were suspended in 500 mL PBS pH 7.4 with gentle stirring to simulate the infinite sink condition. A 25 uL aliquot was collected at different time intervals and replaced with 25 uL of PBS buffer from the incubation medium. The released drug was quantified using UV-Vis Spectroscopy and plotted as cumulative release.

Drug Internalization Assay: RAW 264.7 Macrophage cells were seeded in 10 ml petri dishes. Free BLZ-945 (10 μM) and SHP099 (10 μM) and equivalent amount of dual-inhibitor loaded nanoparticles were added and incubated for 20 h in 5% CO2 atmosphere at 37° C. Following this, the cells were washed with PBS and fresh media was replenished. Once cells reached confluency, they were lysed, centrifuged and supernatant was collected. The amount of drugs in the sample was quantified using UV-Vis Spectroscopy.

Cryo-TEM for DNTs: Nanoparticle samples were preserved using a Vitrobot and liquid ethane supported on plasma-treated lacey carbon 400-mesh copper grids. 5 μL of nanoparticle suspension was applied onto the plasma treated grids, subsequently blotted with filter paper and processed by vitrification in liquid ethane. Electron microscopy was performed using a Phillips CM 120 Cryo operating at 120 keV using a Gatan Oris 2 k by 2 k CCD camera system. Vitreous ice grids were transferred into the cryo-electron microscope using a cryostage that maintains the grids at a temperature below −180° C. Images were acquired at 66 kx (0.152 nm pixels) under low-dose conditions at 8-10 electrons Å².

Flow cytometry to quantify the toxicity of nanoparticles on macrophages: The RAW 264.7 cells were seeded in 12 well plates (8×10⁴ cells per well) and incubated with free drugs or dual-inhibitor loaded nanoparticles at varying concentrations (10 nM-1 μM) at 37° C. for 48 h. Following this, the cells were washed with PBS and collected. The cells were treated with APC Annexin-V (Bio Legend) and Propidium iodide (Bio legend) as per manufacturer's protocol. The cell suspension was transferred to FACS tubes and analyzed for Annexin-V and PI staining on ACEA Novocyte flow cytometer. Data were analyzed using NovoExpress 1.2.5.

In-vitro Phagocytosis Assay: B16/F10 melanoma cells and 4T1 breast cancer cells were labelled with CSFE as per the manufacturers protocol. 2.5×10⁴ B16/F10 and 4T1 cells were taken per well in an ultra-low attachment non-adherent 96 well plate and cocultured with 5×10⁴ M2 polarized (20 ng/ml of IL-4) macrophages that were stained far red at a ratio of 1:2. They were incubated with dual-inhibitor and single-inhibitor loaded nanoparticles or vehicle for 4 h in serum free media. The cells were collected and analyzed by an ACEA Novocyte flow cytometer. Percentage of phagocytosis was determined by the percentage of CSFE positive cells within far red stained macrophage cell gate.

Nitric Oxide estimation assay: 50×10³ RAW 264.7 were seeded in each well of an 8 well chamber slide. They were polarized to M2 phenotype by treatment with 20 ng/ml mouse recombinant IL-4 for 24 hours. Following this, the cells were treated with 500 nM of dual inhibitor loaded nanoparticle and equimolar amounts of free drugs in 5% FBS in DMEM for 48 hours. The cells were washed with PBS and incubated with 10 μM of 4,5-Diaminofluorescein diacetate for 1 hour. Following this, the cells were washed with PBS and fixed with 4% PFA. After subsequent washing, the tissues were counter stained with DAPI and mounted and imaged using a Nikon AlR-SIMe confocal microscope at 20× and analyzed with MS Elements 4.6. Images were adjusted for brightness and contrast.

Flow cytometry assay to quantify repolarization of M2 macrophages to M1 macrophages after inhibition of CSF-1 signaling and SHP2: RAW 246.7 macrophages/BMDMs/Human primary macrophages were seeded at a density of 80×10⁴ cells/well and allowed to reach sub-confluency. They were polarized to M2 phenotype by treatment with 20 ng/ml mouse recombinant IL-4 for 24 hours. Following this, the cells were treated with 500 nM of dual and single inhibitor loaded nanoparticle and equimolar amounts of free drugs in 5% FBS in DMEM for 48 hours. The cells were then washed with PBS, centrifuged and triple stained for Pacific blue CD11b (pan macrophage marker), FITC CD80 (M1 marker) and APC CD206 (M2 marker) or CD163 (M2 marker). Post staining, cells were quantified using ACEA Novocyte flow cytometer and analyzed using NovoExpress 1.2.5 software.

Isolation of Bone Marrow Derived Macrophages: Femur and Tibia were harvested from C57BL/6 mice and the bone marrow was flushed out into a petri plate using PBS. The marrow was collected, centrifuged and resuspended in DMEM media. To this 20 ng/mL of MCSF was added and the cells were incubated for 7 days in 37° C. while the media was replenished every alternate day. At the end of 7 days, the BMDMs would have adhered and these were then polarized to a M2 phenotype by further incubating the with 20 ng/mL of IL-4 for 24 h.

Isolation of human peripheral blood monocyte derived Macrophages: Human blood from donors were collected and various components of blood were separated using a Ficoll density gradient. PBMCs were then isolated and washed twice with RPMI 1640 and were seeded in serum free RPMI 1640 at a seeding density 1 million cells/ml. After 24 h of incubation, media was removed and the non-adherent cells were washed off, after which the cells were incubated in fresh RPMI 1640 supplemented with 10% FBS and 20 ng/ml human MCSF. After 7 days of incubation, 50% excess amount of supplemented RPMI 1640 was media was added. After 3 days of incubation, the media was removed, and the cells were washed with PBS. The cells were then incubated with fresh RPMI 1640 supplemented with 10% FBS and 20 ng/ml human IL-4 for 24 h to obtain M2 human macrophages.

Western Blot Assay to study inhibition of CSF-1R and MAPK pathways at different time points: 5×10⁵ RAW264.7 cells were seeded into 5 mL plates. The cells were incubated with different treatments. After 4 hours of treatment, the cells were washed once with PBS and then replenished with complete media followed by 12 hour and 36 hour-incubation periods at 5% CO₂ and 37° C. Cells were washed after the timepoints and lysed using RIPA lysis buffer while being supplemented with protease and phosphatase inhibitors. BCA assays were performed to measure the amount of protein and equal amount of protein lysates were electrophoresed on a 10% polyacrylamide gel which was then transferred to a PVDF membrane followed by blocking in TBST-T (5% skim milk). Membranes were incubated in 1% BSA in TBST with phospho-CSF1R (1:1000 dilution), CSF1R (1:1000 dilution), SHP-2 (1:1000) and beta-actin (1:2000 dilution) antibodies (Cell Signaling Technology) overnight at 40° C. After washing with TBST, membranes were incubated with horseradish peroxidase-conjugated secondary antibody (1:2000) for 1 hour. Detection was done using Biorad's Clarity ECL (Catalog No.:1705061) and image processing was done by Image J.

Cytotoxic Efficacy of DNTs Using MTS Assay: 5×10³ 4T1 cells or B16 F10 cells per well were seeded in a 96 well plate and left overnight to adhere. The cells were serum starved for 6 h by incubating them in serum free basal medium. The cells were incubated with either free drugs or DNTs of equivalent free drug concentrations for 72 h. The cells were washed with PBS and the medium was replaced with phenol red free DMEM followed by incubation in a mixture of MTS and PMS (1:20) solution for 30 min. Absorbance was measured at 480 nm using a BioTek plate reader. The data was analyzed using graph pad.

Synthesis and Characterization of DNT-206: Anti-mouse CD 206 was purchased from Bio Legend and Fab fragmentation was performed using Pierce F(ab′) 2 fragmentation kit (Thermo Fisher). The F(ab′)2 fragments were collected and concentrated using amicon ultra centrifugal filters (MWCO 3 KDa) and the antibody concentration was established using BCA assay. F(ab′)2 fragments were reduced to Fab nanobodies by incubation the antibodies in MEA at 4° C. The reducing agent was removed by passing the antibodies through a desalting column to obtain thiol presenting nanobodies. DNTS supramolecules were synthesized using the lipid-film hydration method of the following co-lipids 5% of CSF-1R inhibiting amphiphile, 5% of SHP2 inhibitor, 30 mol % of 1,2-distearoyl-sn-glycero-3-phosphoethanolamine-N- [Maleimide (polyethylene glycol)-2000] and 60 mol % L-α-phosphatidylcholine. The CD206 nanobodies were made to attach to the maleimide head group presented by the DNTS nanoparticles by thiol-maleimide reactive chemistry. Unreacted CD206 nanobodies were removed by passing the solution through a sephadex column.

Efficacy Study of DNTs on Murine B16/F10 Melanoma Model: B16/F10 Melanoma cells (1×10⁶) were implanted subcutaneously in the flanks of 4-6 weeks old C57BL/6 mice (weighing 20 g, Charles River Laboratories). When the tumor volume reached ˜100 mm³, the drug therapy was started. The start date of drug therapy was considered as day 0. The therapy consisted of administration of DNT (20 mg/kg) intra venous, DNT 206, BLZ-SNP, SHP2-SNP, SHP099, and Vehicle. The tumor volumes and body weights were monitored on every alternate day for 12 days after injection. The tumor volume was calculated by using the formula, L×B2/2, where the longest diameter was considered as L and the shortest diameter as measured using a Vernier caliper as B. The tumors were harvested immediately following sacrifice and processed for further analysis.

Flowcytometric analysis of excised tumor samples: Harvested tumors were minced and treated with 1 mg/ml type I collagenase and incubated for 1 h at 37° C. and 5% CO². The cell suspension was passed through a 40 μm filter to remove debris and to obtain a single cell suspension. The cells were washed twice with DMEM and the pellet was redistributed into different groups in FACS staining buffer and staining was performed. Post staining, the samples were washed twice to remove unbound antibodies and analyzed using flow cytometry.

Western blot analysis of tumors: Excised tumors were flash frozen in −80° C. The tissues were resected and homogenized using a tissue homogenizer in ice cold NP-40 cell lysis buffer containing protease and phosphatase inhibitors. The lysate was centrifuged at 15000 RPM to remove cell debris. The supernatant was collected, and the protein concentration was estimated using BCA assay. 45 μg of protein was loaded in each well and probed for phospho-CSF-1R, total CSF-1R, phosphor-ERK, total ERK, SHP2, p-SHP2, and beta-actin. Biorad's Clarity ECL was used for detection and image analysis was done by ImageJ.

Ex Vivo Granzyme B estimation: Tumor lysate equivalent to 45 μg of total protein was taken and the volume was adjusted to 50 μL using the assay buffer provided by the Granzyme B activity assay kit (abcam). The activity of granzyme B in the samples was estimated using the manufacturer's protocol.

Immunofluorescent staining for imaging CD45⁺CD8⁺ T cell infiltration: Harvested tissues from each treatment group was flash frozen OCT. 5 μm thin section were obtained using a microtome. The sections were fixed with acetone and blocked with 1% BSA in PBST for non-specific binding. The tissues were incubated at 4° C. overnight with rabbit anti-mouse CD45 (abcam) and rat anti mouse CD8a (BioLegend) primary antibodies. The tissues were washed thrice with PBS and incubated for 1 h at RT with alexa flour 647 goat anti-rat IgG and alexa flour 594 goat anti-rabbit IgG. After subsequent washing, the tissues were counter stained with DAPI and mounted and imaged using a Nikon A1R-SIMe confocal microscope at 20× and analyzed with NIS Elements 4.6. Alexa Flour 647 dye was pseudo-colored green, and alexa flour 594 dye was pseudo colored red. Images were adjusted for brightness and contrast.

Histopathology and TUNEL assay: Harvested tissues (tumors and organs) were flash frozen in OCT. 5 μm thin sections were obtained. Sections were stained with alexa flour 594 Click-iT plus TUNEL assay kit (Thermo Fisher) as per manufacturer's protocol. The nuclei were counter stained with DAPI and subsequently the sections were mounted and imaged using a Nikon A1R-SIMe confocal microscope at 20× and analyzed with NIS Elements 4.6.

Immunofluorescent staining for imaging Ex Vivo Phagocytosis: Harvested tissues from each treatment group was flash frozen OCT. 5 μm thin section were obtained using a microtome. The sections were fixed with acetone and blocked with 1% BSA in PBST for non-specific binding. The tissues were incubated at 4° C. overnight with Rat anti-mouse CD47 (Biolegend) and Rabbit anti mouse CD11b (abcam) primary antibodies. The tissues were washed thrice with PBS and incubated for 1 h at RT with alexa flour 647 goat anti-rat IgG and alexa flour 594 goat anti-rabbit IgG. After subsequent washing, the tissues were counter stained with DAPI and mounted and imaged using a Nikon A1R-SIMe confocal microscope at 20× and analyzed with NIS Elements 4.6. Alexa Flour 647 dye was pseudo-colored green, and alexa flour 594 dye was pseudo colored red. Images were adjusted for brightness and contrast.

In Vivo Biodistribution studies: DiR-tagged DNT 206 nanoparticles were prepared nanoparticles were synthesized using the lipid-film hydration method. 4.5% of CSF-1R inhibiting amphiphile, 4.5% of SHP2 inhibitor, 1% DiR dye, 30 mol % of 1,2-distearoyl-sn-glycero-3-phosphoethanolamine-N-[maleimide (polyethylene glycol)-2000] and 60 mol % L-α-phosphatidylcholine was dissolved in 1 ml of DCM. The DCM was evaporated to obtain a thin film following which, the film was hydrated in PBS at 60° C. for 1.5 h. The nanoparticles were then extruded and incubated with CD206 nanobodies. DNT 206 nanoparticles were administered via tail vein injection to tumor bearing C57BL/6 mice. The imaging was performed at 2 h, 8 h, 12 h, and 24 h post-injection utilizing IVIS filter set (excitation 745 nm and emission 800 nm). All the images were taken using a Perkin Elmer IVIS Spectrum CT In Vivo Imaging system. Exposure times for acquiring data were kept the same, and the stage settings and fiber optic adjustable illuminator arms were kept the same for the duration of observation. Fluorescence signals were normalized and quantified using Living Image 4.7.2 and was used to conduct spectral unmixing. Spectral unmixing was performed by analyzing only the mice in the field of view and using automatic spectral unmixing for each data set to eliminate autofluorescence due to the stage and the mice, leaving fluorescent intensities driven only by the DiR dye itself.

Treatment with FITC-Cholesterol-incorporated DNTs for TAM internalization: 4T1 murine breast cancer cells were injected subcutaneously (1×10⁶ cells) in the right flanks of 4-5-weekold BALB/c mice (weighing between 16-20 g, Charles River Laboratories). Once the tumors reached ˜500 mm³, mice were injected intra-venously with FITC-cholesterol incorporated DNTs (2.5 mg/kg FITC-Chol loading) and sacked 24 hours following. Cryo-sections were taken of the samples and stained with DAPI (blue) and anti-CD11b (red) in order to signify TME cells or macrophages, respectively.

Phagocytosis assay with multi-CD47 protein: B16/F10 melanoma cells were labelled with CFSE as per the manufacturers protocol. 2.5×10⁴ B16/F10 cells were taken per well in an ultra-low attachment non-adherent 96 well plate and cocultured with 2.5×10⁴ M1 polarized (100 ng/ml of LPS and 20 ng/m1 of IFN gamma) macrophages that were stained far red at a ratio of 1:1 They were incubated with 10 ug/ml of CD47 mAb, SIRP alpha mAb, CD47 free protein , multivalent-CD47 or Vehicle for 4 h in serum free media. The cells were collected and analyzed by an ACEA Novocyte flow cytometer. Percentage of phagocytosis was determined by the percentage of CF SE positive cells within far red stained macrophage cell gate.

CD47 binding imaging: The FITC-dye tagged CD47 decorated multivalent supramolecular nanoparticles were synthesized. Briefly, 9 mol % of Cholesterol, 1 mol % FITC-cholesterol conjugate, 30 mol % DSPE-PEG-Carboxy, and 60 mol % L-α-phosphatidylcholine were dissolved in 1 mL of DCM, followed by formation of the thin film by evaporation of DCM. The lipid film was hydrated to form the supramolecular nanoparticles. CD47 protein was then conjugated to the surface at a concentration of 40 ug/ml. The amount of FITC-tag present was determined using a fluorescence spectrometer. RAW264.7 cells were seeded at a density of 50×10³ cells per well onto an 8-well chamber slide. The FITC-tagged nanoparticles were then added at 10 μM (dye concentration) and incubated for different time points. After incubation, the cells were washed with PBS to remove the particles that were not internalized. Cells were then fixed using 4% PFA and stained with DAPI followed by mounting using Invitrogen Glass Antifade reagent. Images were taken at 60× using a Nikon A1R-SIMe confocal microscope and were analyzed using MS Elements 4.6. DSN-treated macrophages were compared to un-treated cells for quantifying the amount internalized in the macrophages.

CD47 binding assay: 100,000 RAW 264.7 macrophages were plated in a 12 well plate. The cells were either incubated with 10 ug/ml of CD47 protein or left untreated for 24 h. The cells were then harvested and stained for the expression of SIRP alpha. The cells were collected and analyzed by an ACEA Novocyte flow cytometer. The expression levels of SIRP-alpha was found to lower in the case of CD47 treated macrophages showing that free CD47 protein indeed binds to SIRP alpha.

### Results/Discussion

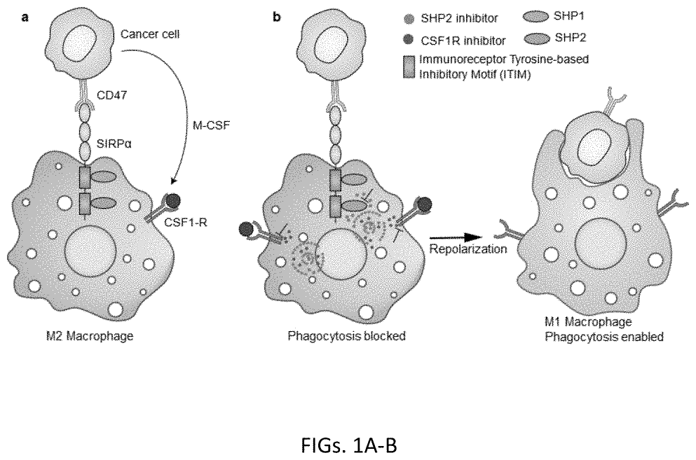

Simultaneous and sustained co-delivery of drugs can be challenging. Combination strategies involving free drug systems are largely ineffective due to limited bioavailability at the tumor site owing to different physiochemical properties and poor solubility. Limited drug availability in the tumors calls for higher dosage of drug administration which increases the susceptibility for systemic toxicities.^{31,32} Moreover, recent studies have highlighted the synergistic importance of deterministic co-drug delivery of drugs in the same cell over stochastic drug distribution in different cells.³³ Hence, in order to overcome these challenges, a self-assembled dual-inhibitors nanotherapeutics (DNTs) loaded with a SHP-2 inhibitor and a CSF1-R inhibitor was designed. It is demonstrated herein that these dual inhibitors nanotherapeutics can internalize into macrophages and co-deliver both inhibitors thereby target both the pathways simultaneously. Furthermore, DNTs exhibited minimal toxicities to macrophages and were able to inhibit both CSF1R and SHP2 in a sustained manner. Concurrent inhibition of both the pathways resulted in successfully skewing the activation states of TAMs towards a more M1 phenotype, while simultaneously enhancing the phagocytic index (FIG. 1). In vivo studies demonstrated that DNTs were able to home into the tumor microenvironment and were able to exhibit sustained inhibition of the CSF1-R and SIRPα signaling axes which in turn correlated to enhanced repolarization of macrophages leading to superior anti-cancer efficacy in aggressive breast cancer and melanoma models, while no significant effects were observed in free drug treatments.

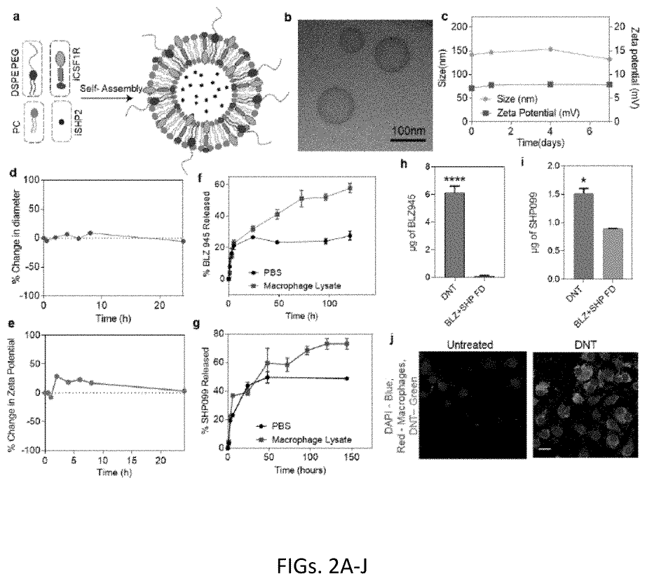

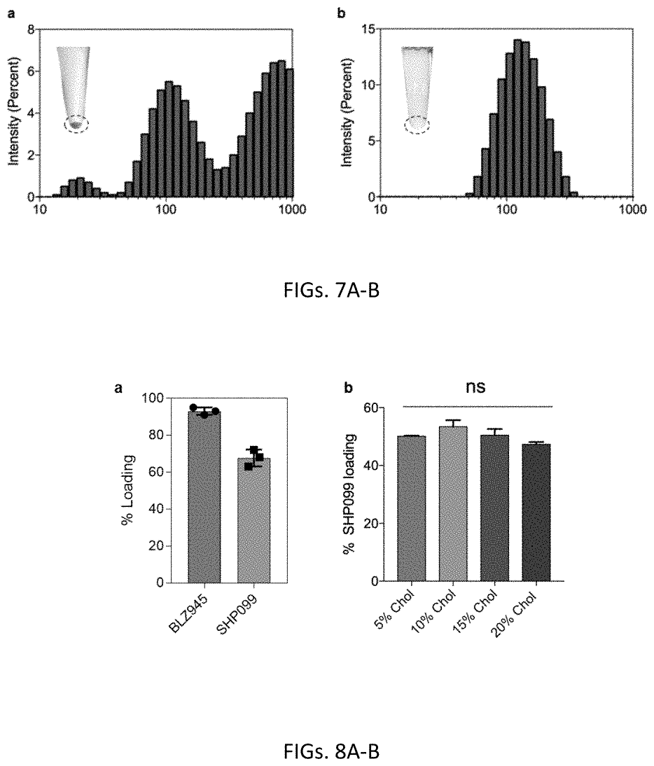

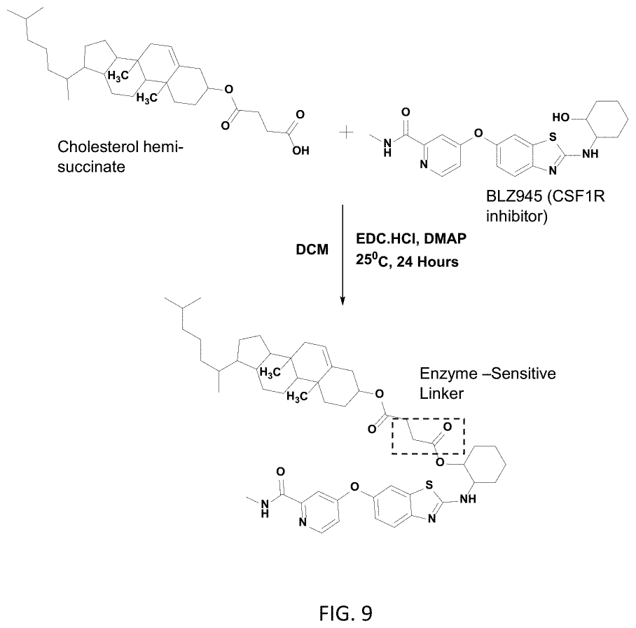

DNTs were envisioned as a dual drug incorporated supramolecular nano-assembly incorporating a CSF1-R inhibiting amphiphile rationally combined with a SHP2 inhibitor. The CSF1-R inhibiting amphiphile was designed using computational algorithm reported in previous studies.^{21,34} The CSF1-R inhibiting amphiphile and SHP2 inhibitor along with co-lipids were able to form a stable supramolecular assembly facilitated by hydrophilic-hydrophobic interactions (FIG. 2A). The selection of the co-lipids, mainly phosphatidyl choline (PC) was rationalized on the basis of increased systemic biocompatibility reported for PC.³⁵ DSPE-PEG was chosen to PEGylate the nanoparticles, which acts like a “cloak” to increase residence circulation time.³⁶ Additionally, DSPE-PEG can be functionalized to present a maleimide head group which can facilitate the attachment of CD206 antibody fragments on the supramolecular surface. Traditional liposomal delivery systems have limited drug loading capacities and previous attempts at encapsulating high drug concentrations have failed.^{34,37} However, supported by results obtained in previous studies and further demonstrated in FIG. 7A-B more than 10 mol % of amphiphiles were incorporated stably into these supramolecular lipid nanoparticles, hence offsetting the limited loading capabilities observed in traditional encapsulation methods.^{21,34} Consistent with these observations, DNTs were synthesized with an average loading efficacy of around 93% of the CSF1-R inhibiting amphiphile and 67% of SHP099 (FIG. 8A). As predicted by the computational simulations, the CSF1-R inhibiting amphiphile occupies the hydrophobic lipid bi-layer, while it was predicted that the more polar SHP099 occupies the hydrophilic core. These results were further confirmed by synthesizing SHP2 nanoparticles loaded with SHP099. Briefly, liposomes of different lipid bi-layer widths were synthesized by varying the percentage of cholesterol. As shown in FIG. 8B, the loading of SHP099 was independent of the amount of cholesterol used to anchor the lipid bilayer, proving that SHP099 is indeed incorporated in the aqueous core of the nanoparticles. This is consistent with recent studies, where unilamellar liposomes were synthesized by physical encapsulation of polar compounds into the inner core of the liposomes.³⁸ Cryo Transmission Electron Microscopy analysis revealed nanoparticles with an average size of 90±23 nm (FIG. 2B). Dynamic light scattering readings show that DNTs have a hydrodynamic diameter of 143±34 nm and a surface zeta potential of 7.9 mV. Next, we tested if the DNTs remained structurally stable for extended periods of time by measuring the size and zeta potential at regular intervals. It was observed that structural integrity of DNTS was maintained for over 7 days as measured by size and zeta potential reinforcing the structural stability (FIG. 2C). The stability of DNTs was further tested in conditions mimicking physiological circulation. Studies have shown that protein corona formation on the surface of nanoparticle is due to preferential surface adsorption of blood serum proteins causing its destabilization.³⁹ The DNTs were incubated in PBS containing 10% human serum and observed that there was no significant change in the size and zeta potential of DNTs for over 24 hours, establishing that these nanoparticles are indeed stable in physiological conditions (FIG. 2D, E). The drug release kinetics profiles of these nanoparticles were analyzed. DNTs were incubated in macrophage cell lysate and PBS. The cell lysate mimics the intracellular compartment in terms of pH and enzymatic action while PBS is used as a control to mimic physiological conditions. It was observed that almost 60% of the drugs was released in cell lysate within the first 100 h of incubation while only 20% of the drug was released in PBS (FIG. 2F, G). This can be attributed to the fact that the nanoparticles are pH sensitive and break down in acidic pH. Moreover, the CSF1-R inhibiting amphiphile is synthesized by chemically conjugating BLZ945 with cholesterol using an enzyme sensitive linker (FIG. 9). This linker is cleaved by an enzyme, esterase which is abundant in cells.⁴⁰ The stability of DNTs in physiological conditions and increased drug release in the intracellular compartment of macrophages is a desirable trait which facilitates selective delivery of both inhibitors into the macrophage compartment. This hypothesis was confirmed by experimentally validating if DNTs can in fact co-deliver both CSF1-R inhibiting amphiphile and SHP2 inhibiting drugs simultaneously to M2 polarized macrophages. Briefly, RAW 264.7 macrophages were polarized to a M2 phenotype by incubating with IL4 and were treated with either DNTs or their equimolar free drug concentrations. After treatment, the cells were collected and lysed, and the amount of internalized drug was evaluated. Not only, was it observed that the DNTs were able to co-deliver both the drugs intracellularly, it was also observed that higher accumulation of drugs delivered through a nanoparticle-based system as compared to of free drugs administration (FIG. 2H, I). These observations validate our initial claims that DNTs can internalize into the macrophages and initiate sustained drug release. Internalization of DNTs into macrophages by incubating RAW 264.7 macrophage cells with FITC-Tagged DNTs was confirmed. It was observed that there was significant internalization of the nanoparticles within 4 h of treatment (FIG. 2J).

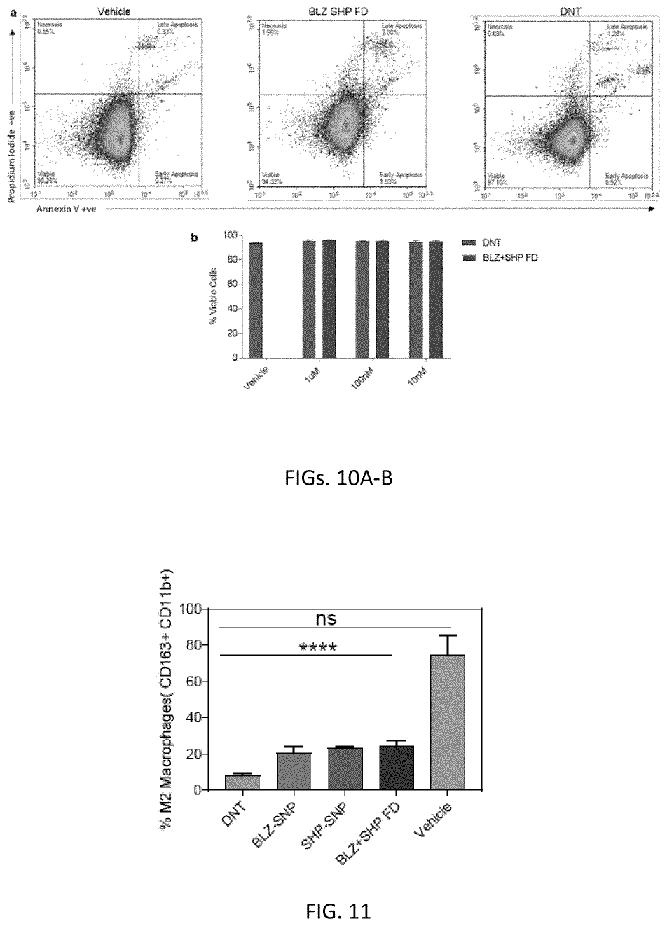

Next, the toxicity profile of these inhibitors on macrophages was studied. SHP2 directly influences the activation of the MAPK pathway, which plays an important role in proliferation of macrophages.⁴¹ Furthermore, the toxicity of the lipid nanoparticle delivery system itself was evaluated as well. RAW 264.7 macrophages were treated with increasing concentration of either DNTs or their equimolar free drug combinations. After 48 h, the cells were harvested and stained for Annexin V and PI. It was observed that both the nanoparticle and free drug combination show minimal cytotoxic effects on macrophages which demonstrate that these inhibitor combinations are indeed nontoxic to macrophages. (FIG. 10A-B). Upon confirmation of the non-toxic toxic nature of these drugs, the role played by DNTs on influencing macrophage polarity and phagocytic capabilities was evaluated.

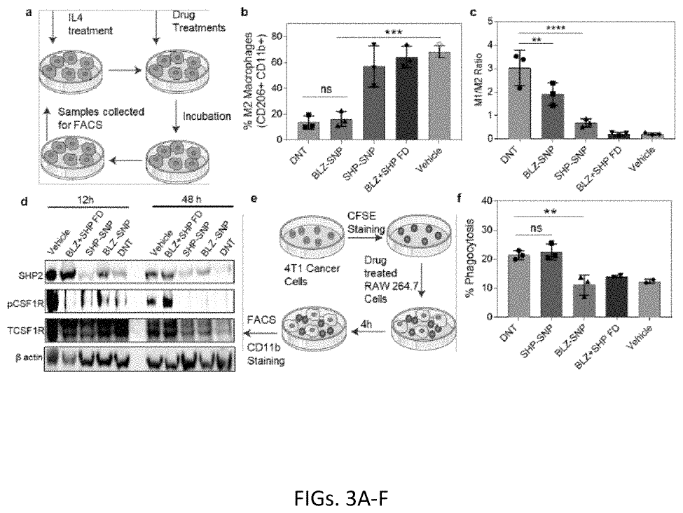

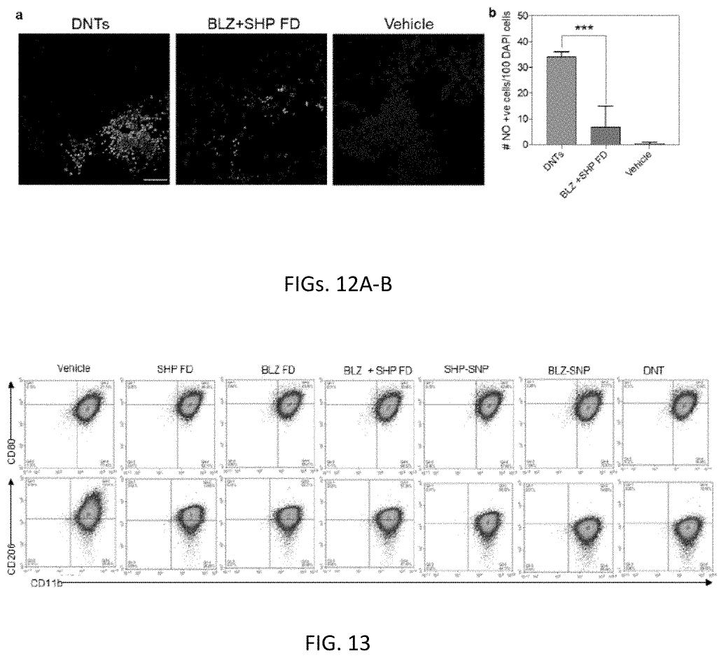

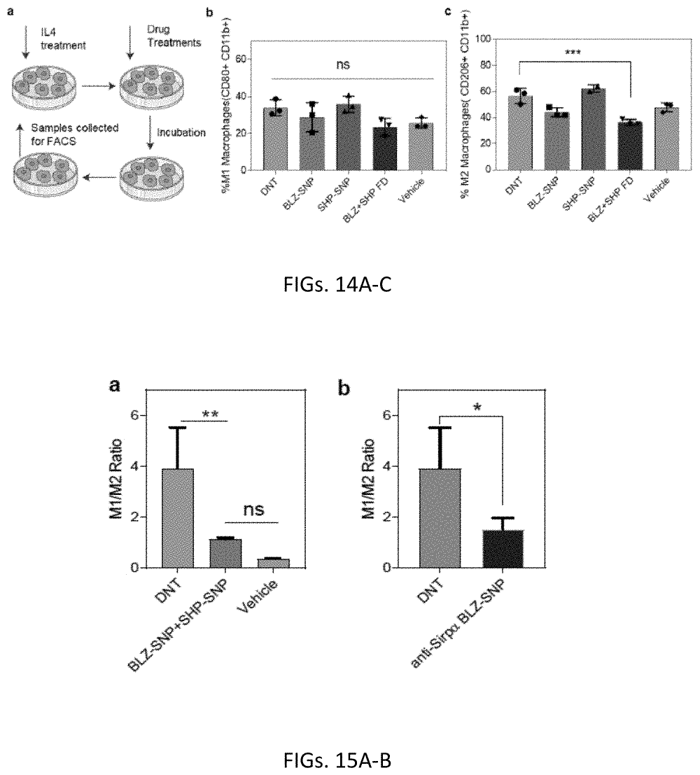

Breast cancer and melanoma cells secrete M-CSF, which polarizes macrophages to an M2 phenotype.⁴²⁻⁴⁵ However, the abundant presence of Th-2 derived cytokines associated with the tumor microenvironment also plays a crucial role in macrophage polarity.⁴⁶ Treatment with the cytokine IL-4 can polarize macrophages to an M2 phenotype in a MCSF independent manner.⁴⁷ Hence, whether sustained inhibition of CSF-1R by DNTs can effectively initiate a M2-M1 switch was tested. As represented in the schematic shown in FIG. 3A, RAW 264.7 macrophages were polarized to a M2 phenotype upon 24 h of treatment with IL-4. Macrophages were then treated with either DNTs or various other drug combinations for different time points. The cells were then harvested and analyzed for M1(CD80+CD11b+) and M2 markers (CD206⁺CD11b⁺).^{48,49} It was observed that treatment with both DNTs and BLZ-SNPs for 48 h results in significant reduction in macrophages with an M2 phenotype and a significant increase in macrophages with an M1 phenotype (FIG. 3B-C). Additionally, we also evaluated the potential of DNTs to downregulate CD163 (FIG. 11), which is regarded as another hallmark of M2 macrophages3 and the ability of DNT macrophages to secrete Nitric Oxide (NO), a hallmark of M1 macrophages⁵⁰was also assessed (FIG. 12) and found that the results obtained post DNT treatment were to be in line with the hypothesis. These results taken together validate previous studies that CSF1-R inhibition could potentially skew macrophage phenotype to an anti-tumorigenic M1 phenotype. However, it is interesting to observe that free drug systems do not effectively repolarize macrophages at longer time points but perform much better at shorter time points (FIGS. 13 and 14A-C). This could be attributed to the fact that sustained inhibition of the CSF1-R signaling axis is absent in free drug systems owing to their poor cellular uptake. Sustained release of CSF1-R inhibiting amphiphile over an extended period of time allows for extended inhibition of CSF1-R. Western blots shown in FIG. 3D further validates this hypothesis. Sustained inhibition of both CSF1R and SHP2 in macrophages treated with DNTs was observed, whereas signal inhibition in free drug systems was only observed at earlier time points. Additionally, it was also observed that co-administration of single drug loaded lipid nanoparticles does not polarize macrophages with the efficiency as compared to DNTs (FIG. 15A). These results are consistent with previous studies demonstrating that in systems where synergistic inhibition of kinase signaling pathways is required, delivery of two different drugs in the same nanoparticle proves to more effective than delivery of the individual drugs in two different nanoparticles. Spatial distribution of both the drugs into the same cellular compartment ensures that both immunosuppressive pathways remains inhibited simultaneously, thus validating our rationale for simultaneous delivery and inhibition of both SHP2 and CSF1R signaling pathways in the same ce11.^{33,51}. Next, the efficacy of DNTs with anti-SIRPα antibody conjugated CSF1R-inhibitor loaded lipid nanoparticles was compared. As shown in FIG. 15B, it was observed that the DNTs exhibit enhanced repolarization efficacy for longer periods of time as compared to anti-SIRPα targeted CSF1R-inhibitng nanoparticles. This could be attributed to the fact that the SIRPα receptors could be replenished by the macrophages after the targeted nanoparticle treatment leading to limited efficacy at longer time periods whereas sustained inhibition of SHP-2 and CSF1R pathway using DNTs results in enhanced repolarization efficiency in vitro.

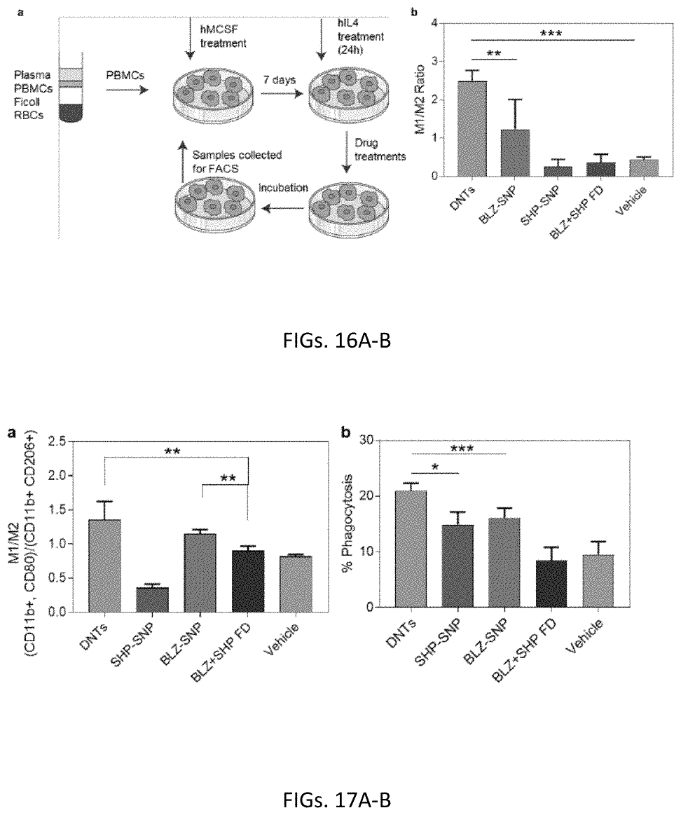

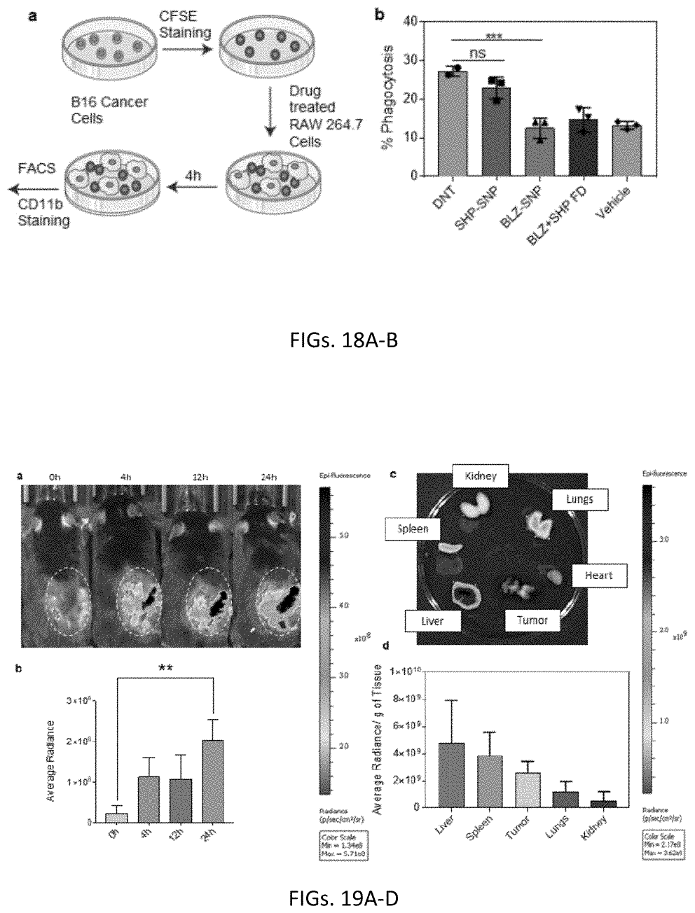

In order to evaluate the translational potential of this system, the repolarization efficacy of DNTs on primary mouse macrophages and primary human macrophages isolated from peripheral blood mononuclear cells (PBMCs) was tested. As shown in FIG. 16 and FIG. 17, it was observed that DNTs are indeed capable of exhibiting amplified macrophage activation and improved phagocytosis. The effect of DNTs on the phagocytic index of macrophages was next evaluated. The SIRPα-CD47 signaling axis plays a crucial role in regulating phagocytosis. Interactions of SIRPα and CD47 promote the tyrosine phosphorylation of the cytoplasmic region of SIRPα, which in turn recruits SHP1 and SHP2 phosphatases.⁵² These set of events lead to the downstream signaling cascades which negatively affects the phagocytic index of the macrophages. It was hypothesized that inhibiting SHP2 in macrophages would halt the downstream signaling cascade associated with inhibition of phagocytosis. As shown by the schematics in FIG. 3E and FIG. 18A, M2 polarized macrophages were subject to different treatment and then co-cultured with either 4T1 breast cancer or B16/F10 melanoma cell lines for 4 h. The macrophages were stained with APC CD11b and the cancer cells were stained with CSFE. The phagocytic index was determined by evaluating the number of double positive cells (Red and Green). Consistent with the initial hypothesis, it was observed that macrophages treated with DNTs and SHP2-SNPs had significantly higher phagocytic index as compared to the other treatment groups (FIG. 3F, FIG. 18B). These results taken together (FIG. 3B and 3E-F) show that concurrent inhibition of SHP2 and CSF1-R pathways work synergistically to repolarize macrophages from a pro-tumorigenic M2 to an anti-tumorigenic M1 phenotype while simultaneously increasing the phagocytic index of the macrophages.

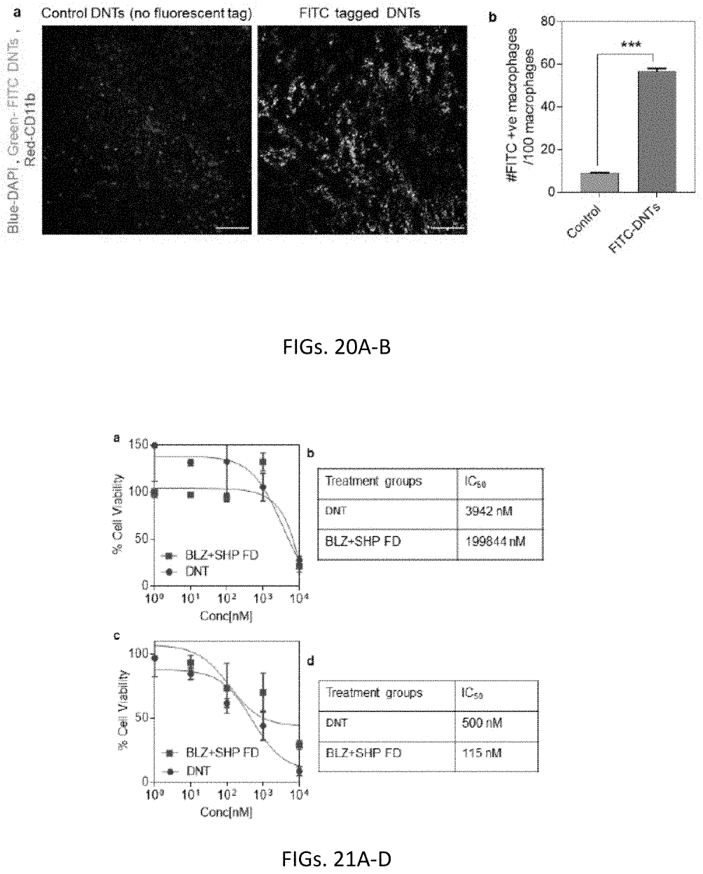

Next, the results obtained in vitro were validated by performing efficacy studies with highly aggressive immunocompetent 4T1 breast cancer and B16/F10 melanoma mouse models. Before evaluating the anti-cancer efficacy of DNTs, the biodistribution of the DNTs was assessed. Tumor-bearing mice were injected with NiR tagged DNTs intravenously through the tail vein and the mice were imaged at different time points using the in vivo imaging software (IVIS). It was observed that the DNTs accumulated into the tumor within 4 hours of administration and maximal accumulation was observed within 24 hours. These results are consistent with previous studies where intra tumoral accumulation was observed due to the enhanced permeability and retention (EPR) effect associated with leaky pathophysiology of the tumor vasculature.⁵³ Next, the mice were sacrificed, and the organs were harvested and imaged (FIG. 19). Maximal accumulation was not only observed in the tumor but also within the liver, lungs and spleen. These observations are consistent with the fact that nanoparticles are up taken by the organs of reticuloendothelial system (RES) before reaching the tumor microenvironment.⁵⁴ Additionally, the efficiency of uptake of DNTs by TAMs in the tumor microenvironment was evaluated. Hence, tumor bearing mice were treated with FITC dye-tagged DNTs and the tumors were harvested after 24 h. Tumors were then sectioned, and counter stained with CD11b. It was observed that DNTs were uptake by more than 50% of the macrophages in the tumor microenvironment (FIG. 20).

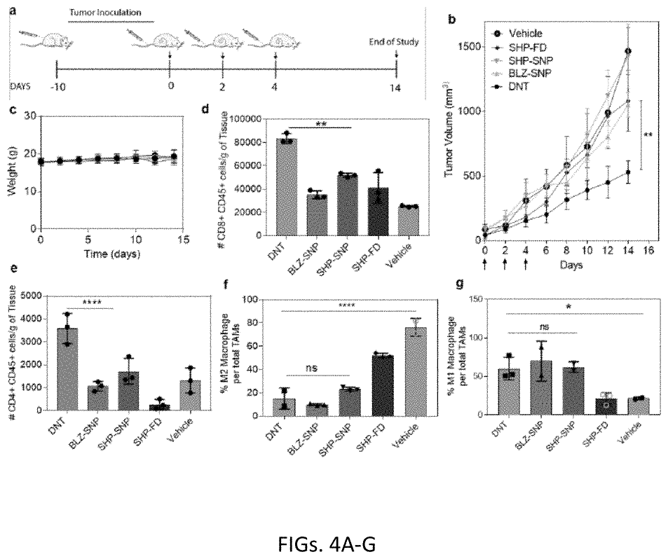

To validate the therapeutic efficacy of the DNTs, BALB/c mice subcutaneously inoculated with 4T1 breast cancer cells. The tumors were allowed to reach a size of ˜75 mm³ and the tumor bearing mice were randomly sorted into different groups. Treatments consisted intravenous administration of sub-optimal doses of either Vehicle, SHP2-SNP, SHP099, BLZ-SNP, DNTs and each dose was normalized to 20 mg kg⁻¹ equivalent of each drug. Optimal doses of 100 mg kg⁻¹ of SHP099 and 200 mg kg⁻¹ were used in previous preclinical monotherapy studies.^{30, 55, 56} But, the reason for using suboptimal doses of 20 mg kg⁻¹ in this study is two-fold. First, to effectively dissect out the combination effect of the drugs in a more concise manner. Second, from the in vitro studies, it was observed that lower concentrations of SHP099 did not have a significant effect on the viability of cancer cells (FIG. 21). Hence, by using sub-optimal doses, treatment efficacy arises as a result of macrophage modulation and not as a result of drug action on cancer cells was effectively concluded. As represented in the schematics in FIG. 4A, the 1st cycle of injection was considered to be day 0, and treatments were administered every alternate day for a total of 3 doses. SHP099 (in DMSO) was administered intraperitoneally. Mice treated with the vehicle developed significantly larger tumors within 10 days of 1st dose, while the tumor growth was significantly inhibited in the groups treated with DNTs (FIG. 4B). Changes in the body weights were within the acceptable limits validating the in vitro data pointing to the non-toxic nature of the treatments (FIG. 4C). At the end of the study, the mice from all the groups were sacrificed at the same time and the tumors were harvested and analyzed further for ex vivo mechanistic studies.

In order to evaluate the mechanism of DNT action, ex vivo analysis of infiltrated immune cells in the tumor microenvironment was evaluated. Immune profiling for various infiltrating tumors showed significantly higher infiltration of CD8⁺ and CD4⁺ activated T cells in groups treated with DNTs as compared to other treatment groups (FIG. 4D-E). It is also interesting to note that, even though mice treated with SHP2-SNP did not show observable anti-tumor efficacy, immune infiltration analysis reveals that SHP2-SNP treated mice had the highest CD8⁺ T infiltration among the groups treated with single kinase-inhibiting drugs. This is in agreement with recent studies by Xiao et al. which showed that myeloid-restricted ablation of SHP2 restricts tumor growth by promoting infiltrating CD8+ T cells.⁵⁷ Enhanced repolarization of TAMs treated with DNTs was observed. As shown in the graphs in FIG. 4F-G, increased percentage of M1 macrophages (CD45+CD11b+CD80+) and reduction in M2 macrophages (CD45+CD11b+CD206+) in DNT treated tumors was observed. It has been established in recent studies that M1 differentiated TAMs play a crucial role in cytotoxic T cell recruitment and activation in the tumor. Hence, a rational combination of CSF1-R inhibitor with a SHP2 inhibitor can synergistically increase T cell infiltration in the tumor.⁵⁸

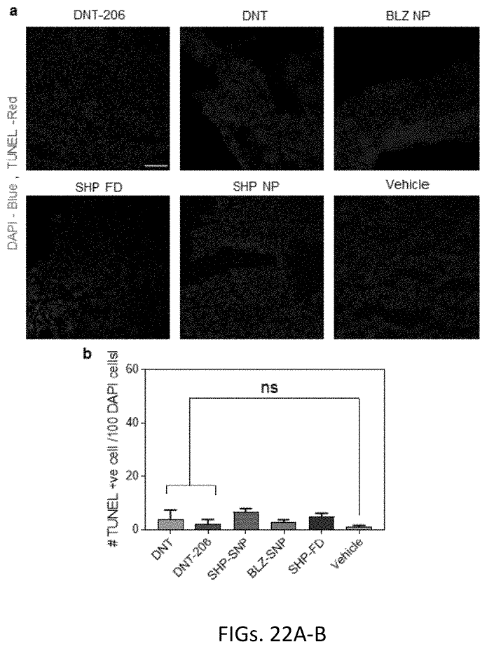

Immunofluorescence staining on excised tissue sections was performed to further support the flow cytometry data pointing to infiltration of activated immune cells in tumor. As shown in FIG. 5A, tumors treated with DNTs showed highest infiltration of CD8⁺ T cells. TUNEL analysis of excised tumor after treatments with different groups was performed next. As shown in FIG. 5B-C, TUNEL analysis on excised tumors was consistent with the tumor progression data. Specifically, it was observed that there was significant cell death in tumors treated with DNTs as compared to other treatment groups. Moreover, isolated liver sections which were also stained for TUNEL showed no significant cell death, further proving that DNTs showed no significant systemic toxicities (FIG. 22). Comprehensive western blot analysis of excised tumor samples shows that the CSF1-R axis and SHP2 pathways are significantly inhibited in DNT treated tumors (FIG. 5D). Densitometry quantifications of pCSF1R and SHP2 revealed significant inhibition of these proteins (FIG. 5E, F). Additionally, there was significant inhibition of phosphorylated ERK1/2 in groups treated with DNTs. Recent studies have shown that the MAPK pathway plays a role in macrophage activation and proliferation.⁵⁹⁻⁶¹ The apparent role of SHP2 inhibitors in regulating ERK inhibition has been previously studied in cancer cells, but it has been relatively unexplored in macrophages.

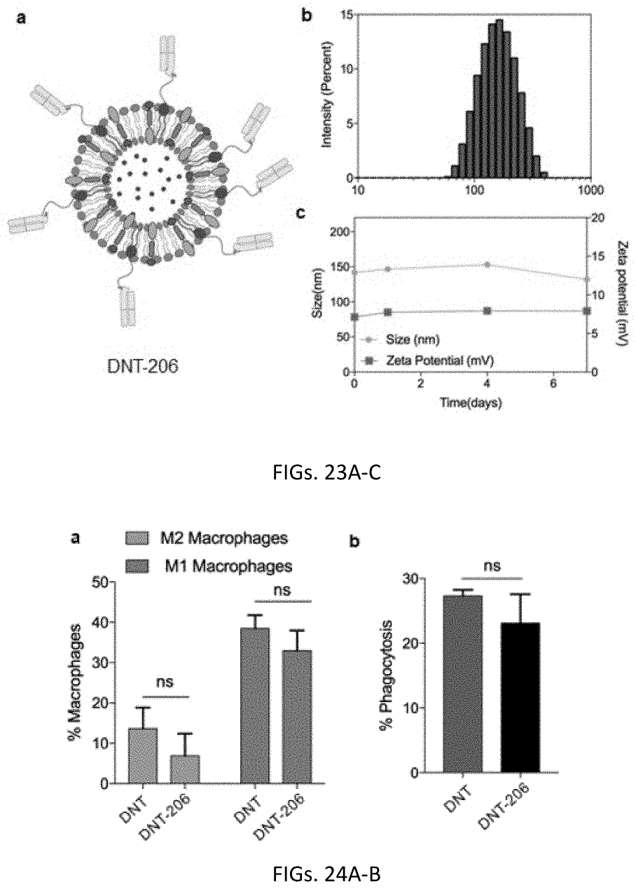

Targeted delivery of drugs to tumor-associated macrophages could improve repolarization efficiency as compared to a non-targeted strategy. Recent studies have also pointed out the potential advantages of enhanced targeting of TAMs using monoclonal CD206 antibodies.⁶²⁻⁶⁴ Hence, the modular design of anti-CD206 DNTs is reported, which can preferentially target TAMs to achieve better repolarization. As represented in the schematics shown in FIG. 23A, a Fab fragmentation kit was used to synthesize anti CD206 Fab fragments. Next, the di-sulfide linkages connecting the 2 fragments were reduced using MEA to obtain Fab′-CD206 fragments with a reactive thiol group. DNTs were synthesized using a modified PEG containing a maleimide head group. The Fab′-CD206 fragments were then conjugated to the surface of the DNT following maleimide-thiol reactive chemistry to obtain DNT206. Size and surface zeta potential measured over a period of 7 days revealed that these DNT206 were stable for extended periods of time (FIG. 23B-C). Before validating the in vivo efficacy of DNTs, the phagocytic and repolarization capabilities of DNTs with anti-CD206 DNTs in vitro were compared (FIG. 24). It was observed that there was no significant difference in efficacies between both the groups which led us to the conclusion that targeting of TAMs using anti-CD206 antibody conjugated DNTs in vitro does not significantly improve macrophage activation and phagocytosis.

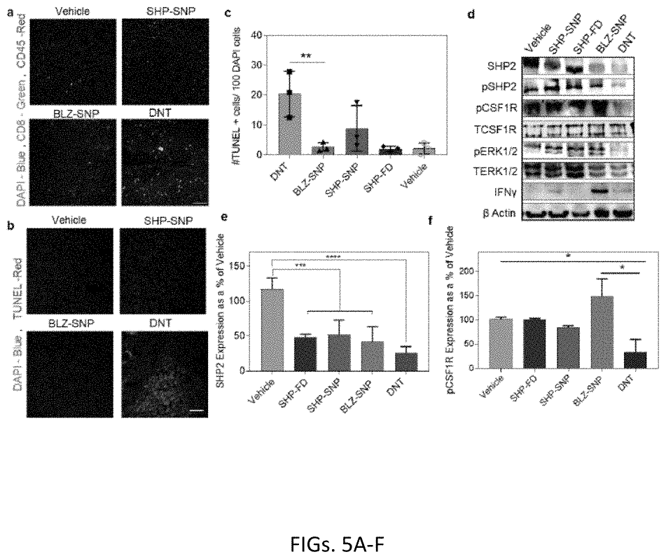

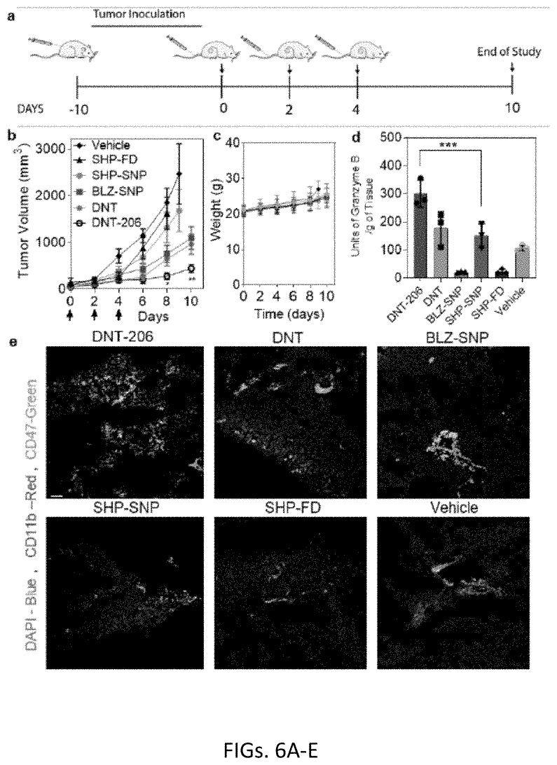

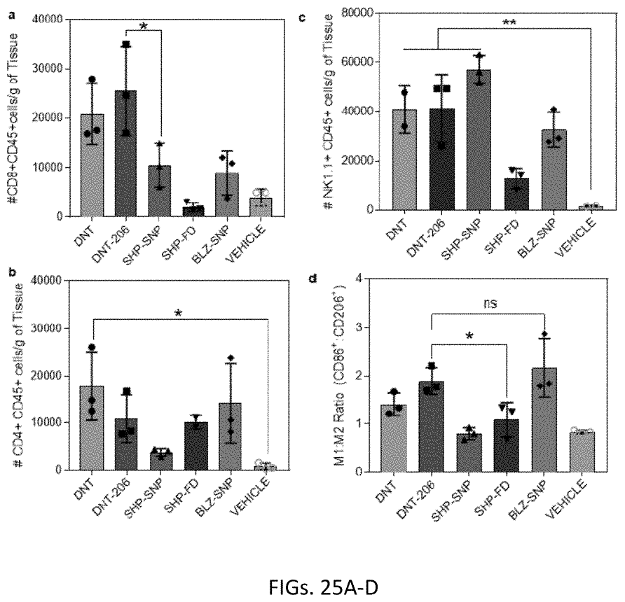

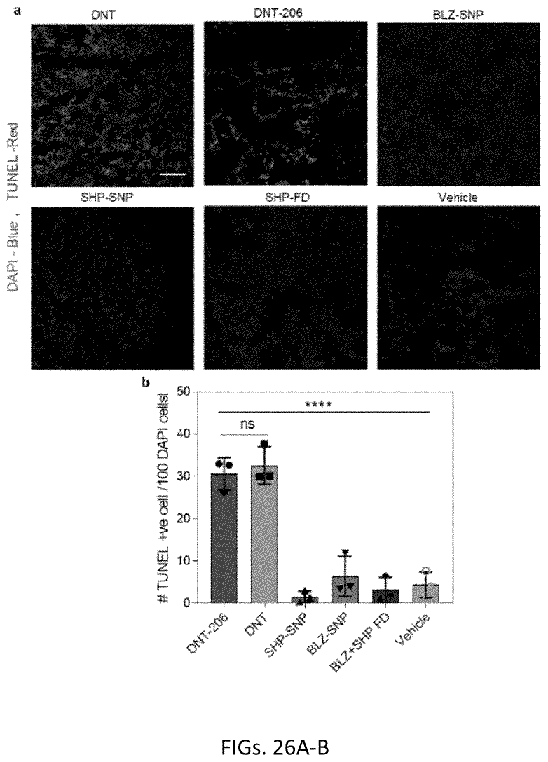

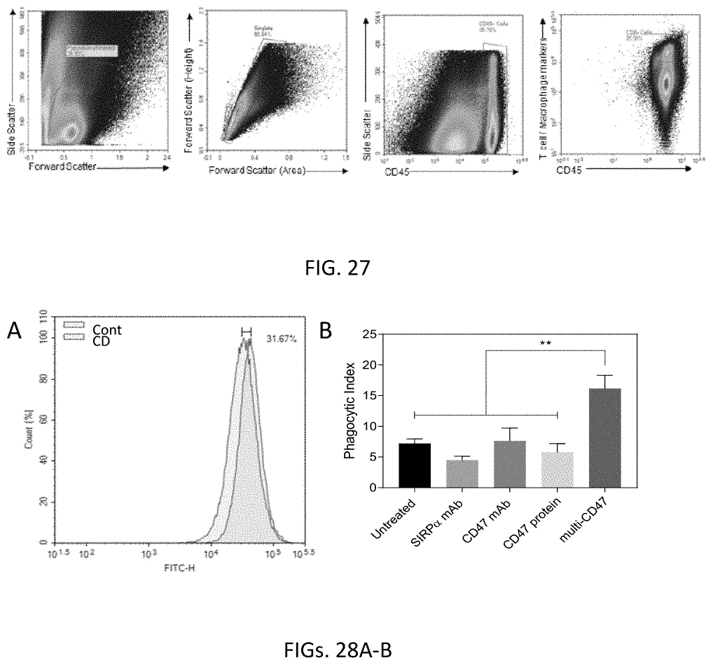

Next, the efficacies of DNTs and DNT206s was validated in a second immunocompetent melanoma bearing C56BL/6 mouse model. B16/F10 melanoma tumor-bearing mice were subjected to the treatment cycle shown in FIG. 6A. Significant tumor reduction was observed in groups treated with DNT206 as compared to other treatment groups (FIG. 6B-C). Consistent with the previous studies, ex vivo analysis of tumor samples revealed an increased infiltration of immune cells, mainly effector T cells (CD4⁺ and CD8a⁺) and NK1.1⁺ cells in groups treated with DNTs and DNT-206 (FIG. 25A-C). A higher incidence of repolarized macrophages in groups treated with DNT-206 as compared to DNTs was observed (FIG. 25D), validating that targeted delivery of DNTs could effectively enhance repolarization. In order to further validate that infiltrating effector T cells were in fact cytotoxic, the activity of granzyme B release was evaluated in excised tissues and was found to be significantly higher in tissues treated with DNT-206 (FIG. 6D). In addition to this, a higher rate of phagocytic macrophages in groups treated with DNT-206 was observed. This observation is represented in FIG. 6E, where tumor sections fluorescently stained with a cancer cell marker (CD47) and a macrophage marker (CD11b) showed much higher signal colocalization in groups treated with DNT-206 as compared to other groups. TUNEL assay performed analysis on excised tumors is also consistent with the tumor progression data, where it was observed that there was significant cell death associated with tumors treated with DNTs and DNT206s (FIG. 26).

In conclusion, it has been demonstrated that dual inhibitors loaded nanotherapeutics (DNTs) can be synthesized by stably incorporating a CSF1-R inhibiting amphiphile and a SHP2 inhibitor facilitated by the self-assembly of co-lipids. These DNTs that deterministically co-deliver both inhibiting amphiphiles in the same cells results in concurrent inhibition of both the CSF1-R and CD47-SIRPα signaling pathways. Sustained release of these inhibitors after uptake by the M2 macrophages results in its enhanced repolarization to activated M1 macrophages with simultaneous increase in its phagocytic functions. These results were further validated by in vivo tumor efficacy studies performed in aggressive 4T1 breast cancer mouse model, where improved anti-cancer efficacy was observed in mice treated with DNTs. The mechanism of action was further validated with ex vivo studies showing significant inhibition of CSF1-R and SHP2 pathways and superior immune activation leading to enhanced effector T cells infiltration. Additional studies involving DNT206s used for specifically targeting M2 macrophages in Bl6F10 melanoma mouse model revealed even higher anti-tumor efficacies.

### BIBLIOGRAPHY

1. C. E. Lewis, J. W. Pollard, Cancer research, 2006, 66, 605.
2. S. B. Coffelt, R. Hughes, C. E. Lewis, Biochimica et biophysica acta, 2009, 1796, 11.
3. T. Roszer, Mediators Inflamm, 2015, 2015, 816460.
4. S. Zanganeh, R. Spitler, G. Hutter, J. Q. Ho, M. Pauliah, M. Mahmoudi, Immunotherapy, 2017, 9, 819.
5. N. Wang, H. Liang, K. Zen, Frontiers in immunology, 2014, 5, 614.
6. H. Wang, L. Yang, D. Wang, Q. Zhang, L. Zhang, Hum Vaccin Immunother, 2017, 13, 1556.
7. J. O'Brien, P. Schedin, Journal of mammary gland biology and neoplasia, 2009, 14, 145.
8. C. Engblom, C. Pfirschke, M. J. Pittet, Nature reviews. Cancer, 2016, 16, 447.
9. R. Noy, J. W. Pollard, Immunity, 2014, 41, 49.
10. D. I. Gabrilovich, et al., Nature reviews. Immunology, 2012, 12, 253.
11. S. I. Grivennikov, F. R. Greten, M. Karin, Cell, 2010, 140, 883.
12. E. R. Stanley, V. Chitu, Cold Spring Harb Perspect Biol, 2014, 6.
13. M. A. Cannarile, et al., Journal for immunotherapy of cancer, 2017, 5, 53.
14. L. Cassetta, T. Kitamura, Front Cell Dev Biol, 2018, 6, 38.
15. V. D. Edwards, et al., Oncotarget, 2018, 9, 24576.
16. W. D. Tap, et al., The New England journal of medicine, 2015, 373, 428.
17. H. T. Temple, Current opinion in oncology, 2012, 24, 404.
18. D. F. Quail, et al., Science, 2016, 352, aad3018.
19. M. I. El-Gamal, et al., J Med Chem, 2018, 61, 5450.
20. J. Escamilla, et al., Cancer research, 2015, 75, 950.
21. A. Kulkarni, et al., Cell, 2009, 138, 271.
23. A. N. Barclay, T. K. Van den Berg, Annual review of immunology, 2014, 32, 25.
24. K. Weiskopf, Eur J Cancer, 2017, 76, 100.
25. J. Liu, et al., Journal of clinical oncology: official journal of the American Society of Clinical Oncology, 2019, 37, 946.
27. T. Yanagita, et al., JCI Insight, 2017, 2, e89140.
28. Y. Murata, et al., Cancer Sci, 2018, 109, 1300.
29. Y. Murata, T. Kotani, H. Ohnishi, T. Matozaki, J Biochem, 2014, 155, 335.
30. Y. N. Chen, et al., Nature, 2016, 535, 148.
31. P. Parhi, C. Mohanty, S. K. Sahoo,Drug Discov Today, 2012, 17, 1044.
32. F. Greco, M. J. Vicent, Advanced drug delivery reviews, 2009, 61, 1203.
33. A. Goldman, et al., ACS nano, 2016, 10, 5823.
34. A. Kulkarni, et al., ACS nano, 2016, 10, 9227.
35. F. Bonte, et al., Biochimica et biophysica acta, 1987, 900, 1.
36. J. S. Suk, et al., Advanced drug delivery reviews, 2016, 99, 28.
37. L. Sercombe, et al., Front Pharmacol, 2015, 6, 286.
38. L. B. Lopes, et al., Colloids Surf B Biointerfaces, 2004, 39, 151.
39. P. d. Pino, et al., Materials Horizons, 2014, 1, 301.
40. Y. 0. Yoshimitsu Yamazakia, et al., Biochimica et biophysica acta, 1995, 1243, 300
41. X. J. Li, et al., J Biol Chem, 2015, 290, 3894.
42. P. Pathria, T. L. Louis, J. A. Varner, Trends in immunology, 2019, 40, 310.
43. R. Cui, W. Yue, E. C. Lattime, M. N. Stein, Q. Xu, X. L. Tan, Oncotarget, 2016, 7, 50735.
44. S. Ramakrishnan, et al., The Journal of clinical investigation, 1989, 83, 921.
45. J. Park, et al., Cell Rep, 2015, 10, 1614.
46. D. G. DeNardo, L. M. Coussens, Breast Cancer Research, 2007, 9, 212.
47. M. Genin, et al., Frontiers in immunology, 2017, 8, 1097.
49. A. Kulkarni, et al., Nature Biomedical Engineering, 2018, 2, 589.
50. S. B. Weisser, et al., Methods Mol Biol, 2013, 946, 225.
51. A. Ramesh, et al., Cellular and Molecular Bioengineering, 2019, DOI: 10.1007/s12195-019-00576-1.
52. D. R. Soto-Pantoja, S. Kaur, D. D. Roberts, Crit Rev Biochem Mot Blot, 2015, 50, 212.
53. J. Fang, et al., Advanced drug delivery reviews, 2011, 63, 136.
54. S. Nie, Nanomedicine, 2010, 5, 523.
55. P. A. Cassier, et al., The Lancet. Oncology, 2015, 16, 949.
56. C. Fedele, et al., Cancer Discov, 2018, 8, 1237.
57. P. Xiao, Y. Guo, H. Zhang, X. Zhang, H. Cheng, Q. Cao, Y. Ke, Oncogene, 2018, 37, 5088.
58. G. Genard, S. Lucas, C. Michiels, Frontiers in immunology, 2017, 8, 828.
59. C. I. Caescu, et al., Blood, 2015, 125, el.
60. E. T. Richardson, et al., PloS one, 2015, 10, e0140064.
61. D. Zhou, et al., Cellular signalling, 2014, 26, 192.
62. Y. Singh, et al., Journal of controlled release: official journal of the Controlled Release Society, 2017, 254, 92.
63. A. Al Faraj, et al., International journal of nanomedicine, 2014, 9, 1491.
64. C. Zhang, et al., Biomaterials, 2016, 84, 1.

All publications, nucleotide and amino acid sequence identified by their accession nos., patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims. As used herein and in the appended claims, the singular forms “a,” “an,” and “the” include plural reference unless the context clearly dictates otherwise. Thus, for example, a reference to “a nucleic acid” or “a polypeptide” includes a plurality of such nucleic acids or polypeptides (for example, a solution of nucleic acids or polypeptides or a series of nucleic acid or polypeptide preparations), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

What is claimed is:

1. A composition comprising an engineered therapeutic, wherein the engineered therapeutic comprises at least one inhibitor of the CSF1-R signaling pathway and at least one inhibitor of the CD47-SIRPα signaling pathway.

2. The composition of claim 1, wherein the engineered therapeutic comprises phosphatidyl choline (PC).

3. The composition of claim 2, wherein the PC is L-α-phosphatidylcholine.

4. The composition of claim 1, wherein the engineered therapeutic comprises cholesterol, Sitosterol, 1-Lysophosphatidylcholine or other conjugatable lipids.

5. The composition of claim 1, wherein the nanoparticles are pegylated.

6. The composition of claim 5, wherein 1,2-distearoyl-sn-glycero-3-phosphoethanolamine-N-(amino(polyethylene glycol)-2000) pegylates the nanoparticles.

7. The composition of claim 1, wherein at least one inhibitor of the CSF1-R signaling pathway is CSF1R kinase inhibitor.

8. The composition of claim 7, wherein the CSF1R kinase inhibitor is 4-((2-(((1R,2R)-2-hydroxycyclohexyl)amino)benzo[d]thiazol-6-yl)oxy)-N-methylpicolinamide (BLZ-945).

9. The composition of claim 1, wherein at least one inhibitor of the CD47-SIRPα signaling pathway is an inhibitor of SHP2.

10. The composition of claim 9, wherein the inhibitor of the SHP2 is 6-(4-Amino-4-methylpiperidin-1-yl)-3-(2,3-dichlorophenyl)pyrazin-2-amine dihydrochloride (SHP099).

11. The composition of claim 1, wherein a targeting agent is conjugated to the engineered therapeutic.

12. The composition of claim 11, wherein the targeting agent is an anti-CD206 antibody.

13. The composition of claim 1, further comprising a carrier.

14. A method to treat cancer comprising administering to a subject in need thereof the composition of claim 1.

15. The method of claim 14, further comprising administering an additional anti-cancer agent including a small molecule, antibody, protein, peptide.

16. A method to repolarize M2 macrophages to activated M1 macrophages comprising contacting the M2 macrophage with a composition of claim 1.

17. A method to increase the phagocytic function of a macrophage comprising contacting the macrophage with a composition of claim 1.

18. The composition of claim 1, wherein the inhibitor of the CD47-SIRP signaling pathway is CD47 protein.

19. The composition of claim 1, wherein the inhibitor of the CSF1-R signaling pathway comprises a chemical backbone with known binding affinity.

20. The method of claim 14, further comprising administering an additional anti-cancer agent including a targeted therapy, immunotherapy, chemotherapy, or radiation.
